(19) United States
(12) Patent Application Publication
(10) Pub. No.: US 2026/0217781 A1
(43) Pub. Date: Jul. 30, 2026

(54) PEPTIDE CONJUGATES FOR LABELLING ENDOGENOUS GIPR AND GLP-1R

(71) Applicants: FORSCHUNGSVERBUND BERLIN E.V, Berlin (DE); University of Birmingham, West Midlands (GB)

(72) Inventors: Johannes BROICHHAGEN, Berlin (DE); David HODSON, West Midlands (GB)

(73) Assignees: FORSCHUNGSVERBUND BERLIN E.V, Berlin (DE); University of Birmingham, West Midlands (GB)

(21) Appl. No.: 19/140,669

(22) PCT Filed: Dec. 19, 2023
(86) PCT No.: PCT/EP2023/086605

§ 371 Date: Jun. 18, 2025

(30) Foreign Application Priority Data
Dec. 19, 2022 (EP) 22214640.9

(51) Int. Cl. C07K 14/575; C07K 1/107; C07K 14/605; G01N 33/58; G01N 33/74

(52) U.S. Cl. CPC C07K 14/575; C07K 1/1077; C07K 14/605; G01N 33/582; G01N 33/74; G01N 2333/726

(57) ABSTRACT

The invention relates to a peptide conjugate comprising a peptide covalently linked to a label, wherein the peptide comprises an amino acid sequence of a protein ligand that binds at least one class B1 G protein-coupled receptor, wherein the peptide is covalently linked to the label. The protein ligand is preferably an agonist, antagonist and/or co-agonist of at least one class B1 G protein-coupled receptor, preferably from the Glucagon-like subfamily. The agonist, antagonist and/or co-agonist is preferably selected from tirzepatide, LY3437943 or GIP. The invention further relates to kits comprising the peptide conjugate according to the invention or means for assembling the conjugate, as well as methods for using the peptide conjugate according to the invention.

The invention is in the field of biology, specifically in the field of molecular biology and biochemistry.

The invention relates to a peptide conjugate comprising a peptide covalently linked to a label, wherein the peptide comprises an amino acid sequence of a protein ligand that binds at least one class B1 G protein-coupled receptor, wherein the peptide is covalently linked to the label.

The protein ligand and/or the peptide of the conjugate is preferably an agonist, antagonist and/or co-agonist of at least one class B1 G protein-coupled receptor, preferably from the Glucagon-like subfamily. The agonist, antagonist and/or co-agonist is preferably selected from the group consisting of GLP-1, GIP, glucagon or a precursor protein thereof, or dual and or triple agonists of class B1 G protein-coupled receptors, such as those comprising sequences of GLP-1, GIP and glucagon, or combinations thereof, such as tirzepatide or LY3437943.

The invention further relates to kits comprising the peptide conjugate according to the invention, as well as methods for assembling and using the same.

The invention relates to an in vitro method for labelling and/or detecting the presence and/or localization of class B1 G protein-coupled receptors in a sample. The invention relates to the use of the peptide conjugate of the invention for labelling class B1 G protein-coupled receptors in vitro. The invention relates to a kit comprising: the peptide conjugate of the invention, or a peptide and a label configured for covalent linkage.

### BACKGROUND OF THE INVENTION

Glucose-dependent insulinotropic polypeptide (GIP) is a gut hormone released from enteroendocrine cells lining the proximal small intestine following the ingestion of a meal. GIP is a critical component of the incretin axis and, together with glucagon-like peptide-1 (GLP-1), augments post-prandial insulin release through direct engagement of pancreatic beta cells [1]. Recent advances in pharmacology have highlighted the additional therapeutic benefits of leveraging the extra-pancreatic effects of GIP signaling for the treatment of obesity.

In recent years the GIP receptor (GIPR) has become a promising therapeutic target for the treatment of type 2 diabetes and obesity. Clinical trials have shown remarkable efficacy of dual GIPR and GLP1R agonists. While agonists for the GIP receptor (GIPR) given in isolation elicit modest reductions in body weight [2,3], in pre-clinical and clinical studies, the GIPR signaling axis has proven to be an effective co-target when combined with other anorexic hormones [2, 4-8] [9-12]. For GIP/GLP-1 dual agonism, this potent effect on weight loss correlates with decreased food intake [13, 14], suggesting underlying central mechanisms. Indeed, CNS expression of Gipr is necessary for the synergistic weight loss elicited by GIPR/GLP1R dual agonism, indicating the importance of the GIPR signaling axis in the brain and understanding its role in regulating energy balance [15].

Dual agonists are typically more effective than GLP1R agonists alone, which have been the mainstay of type 2 diabetes and obesity treatment until today. Tirzepatide (Eli Lilly) was FDA approved in 2022 for the treatment of type 2 diabetes and has received fast-track submission for the future treatment of obesity. The efficacy of tirzepatide regarding weight loss has been shown to be comparable to bariatric surgery.

Despite these promising therapeutic applications of targeting GIPR and GLP1R, little is known about GIPR or GLP1R localization in the body, as the targeting of said receptors with specific antibodies has been unsuccessful until today. At the same time, no functional data exists on the interaction and binding of Tirzepatide to both GLP1R and GIPR in vivo, e.g., in tissues expressing one or both receptors. First genetic experiments using mouse models have shown localization of GLP1R and GIPR in the brain and pancreas, however, in said experiments GIPR and GLP1R were not labelled themselves.

In summary, means for specifically targeting and labelling GLP1R and GIPR are still unavailable, which has a negative impact on basic and functional research on this molecular signaling network, which plays an important role in diseases like Diabetes Type 2.

Thus, approaches for specific targeting and/or labelling of GLP1R and/or GIPR are urgently needed and highly desirable, for example as research and drug development tools.

### SUMMARY OF THE INVENTION

In light of the prior art the technical problem underlying the present invention is to provide alternative or improved means for targeting and/or labelling GLP1R and/or GIPR in vitro and in vivo.

This problem is solved by the features of the independent claims. Preferred embodiments of the present invention are provided by the dependent claims.

The invention therefore relates in a first aspect to a peptide conjugate comprising a peptide and a label, wherein the peptide comprises an amino acid sequence of a protein ligand that binds at least one class B1 G protein-coupled receptor, wherein the peptide is covalently linked to the label.

In embodiments the protein ligand and/or the peptide of the conjugate is an agonist, antagonist and/or co-agonist of at least one class B1 G protein-coupled receptor.

In preferred embodiments, the protein ligand is tirzepatide, LY3437943 or Glucose-dependent insulinotropic polypeptide (GIP).

In embodiments the protein ligand is an agonist of at least one class B1 G protein-coupled receptor. In embodiments the protein ligand is a dual or triple agonist of the at least one class B1 G protein-coupled receptor. In embodiments the protein ligand is an antagonist of at least one class B1 G protein-coupled receptor. In embodiments the protein ligand is a dual or triple antagonist of the at least one class B1 G protein-coupled receptor. In embodiments the protein ligand is a co-agonist of at least one class B1 G protein-coupled receptor. In embodiments the protein ligand is a dual or triple co-agonist of the at least one class B1 G protein-coupled receptor.

In embodiments the protein ligand is selected from the group consisting of dual or triple agonists of the at least one class B1 G protein-coupled receptor comprising sequences of GIP, glucagon and/or GLP-1, or combinations thereof, such as tirzepatide, LY3437943 and GIP, or a precursor protein of GIP.

In embodiments the peptide of the present conjugate comprises an amino acid sequence of an agonist, antagonist and/or co-agonist of at least one class B1 G protein-coupled receptor, or comprises an amino acid sequence with at least 70% identity, preferably 80%, more preferably 90% or 95% identity, to said agonist, antagonist and/or a co-agonist, wherein the peptide of the present conjugate is covalently linked to the label.

In embodiments the protein ligand comprises an amino acid sequence with at least 70% identity, preferably 80%, more preferably 90% or 95% identity, to an endogenous human protein ligand of at least one class B1 G protein-coupled receptor.

In embodiments of the peptide conjugate according to the invention, the at least one class B1 G protein-coupled receptor is selected from the Glucagon-like subfamily comprising Glucagon-like peptide 1 receptor (GLP-1R), glucose-dependent insulinotropic polypeptide receptor (GIPR), GLP-2R, GCGR, GHRHR and SCTR.

Despite the recent development of therapeutics targeting GIPR and/or GLP1R, approaches to label and detect these receptors with specific antibodies have not been successful. Accordingly, neither tissue localization of GIPR or GLP1R, nor the exact mechanism of action of GIPR- and/or GLP1R-targeting drugs have been unraveled to date.

Hence, it was entirely surprising that class B1 G protein-coupled receptors, such as GIPR and GLP-1R, can be specifically targeted and labelled with the peptide conjugates according to the present invention.

In embodiments of the peptide conjugate according to the invention, the protein ligand is selected from the group consisting of GIP, glucagon, GLP-1, or a precursor protein thereof, or dual and triple agonists of the at least one class B1 G protein-coupled receptor, comprising sequences of GIP, GLP-1 or glucagon or combinations thereof. Examples of dual and triple agonists are tirzepatide and LY3437943.

In embodiments of the peptide conjugate according to the invention, the protein ligand comprises a combination of sequences from multiple class B1 G protein-coupled receptor agonists, such as tirzepatide or LY3437943.

In embodiments a combination of sequences from multiple class B1 G protein-coupled receptor agonists comprises sequences of GIP, glucagon or GLP-1, or combinations thereof.

In embodiments of the peptide conjugate according to the invention, the agonist, antagonist and/or co-agonist is selected from dual and triple agonists of the at least one class B1 G protein-coupled receptor, comprising sequences of GIP, GLP-1, or glucagon or combinations thereof, such as the dual or triple agonists tirzepatide and LY3437943.

In embodiments of the peptide conjugate according to the invention, the agonist, antagonist and/or co-agonist is selected from the group consisting of GIP, GLP-1, glucagon or a precursor protein thereof, or dual and triple agonists of the at least one class B1 G protein-coupled receptor, comprising sequences of GIP, GLP-1 or glucagon or combinations thereof. Examples of dual and triple agonists are tirzepatide and LY3437943.

In embodiments, the peptide of the peptide conjugate comprises the entire (full-length) or partial amino acid sequence (portion/fragment) of the naturally/endogenously (preferably in human) occurring amino acids sequence of the protein ligand of at least one class B1 G protein-coupled receptor, or of the precursor or pre-(pro-) protein of protein ligand.

In embodiments, the peptide of the peptide conjugate comprises the entire (full-length) or partial amino acid sequence (portion/fragment) of the naturally/endogenously (preferably in human) occurring amino acids sequence of the protein ligand of at least one class B1 G protein-coupled receptor, or of the precursor or pre-(pro-) protein of the protein ligand.

In embodiments, the peptide of the peptide conjugate comprises the entire (full-length) or partial amino acid sequence (portion/fragment) of the naturally/endogenously (preferably in human) occurring amino acids sequence of the agonist, antagonist and/or a co-agonist of at least one class B1 G protein-coupled receptor, or of the precursor or pre-(pro-) protein of the agonist, antagonist and/or a co-agonist.

In embodiments of the peptide conjugate according to the invention, the protein ligand is tirzepatide.

In embodiments of the peptide conjugate according to the invention, the protein ligand is LY3437943.

In embodiments, the protein ligand comprises a sequence of GIP or a fragment thereof, such as tirzepatide, LY3437943 or GIP. Both tirzepatide and LY3437943 comprise a combination of sequences from multiple class B1 G protein-coupled receptor agonists, such as a GIP partial (peptide) sequence. Embodiments of protein ligands comprising a sequence of GIP or a fragment thereof, such as tirzepatide, LY3437943 and GIP itself, are unified by the shared sequence of GIP or fragments (partial sequence of GIP) thereof. In embodiments, said full or partial sequences of GIP enable embodiments of the present conjugates to interact and bind to GIPR thereby facilitating, e.g., the labelling, detection and research of the receptor. The advantageous effects of such conjugates comprising a full or partial sequence of GIP are shown, e.g., in the Examples 1 and 2 and the Figures herein, wherein the surprisingly sensitive and efficient detection and labelling of class B1 G protein-coupled receptors, such as GIPR is evidenced. Embodiments of the present conjugate comprising full or partial sequences of GIP are unified at least by said surprising and beneficial effect shown in the present examples and described herein.

In embodiments of the peptide conjugate according to the invention, the protein ligand is GIP.

In embodiments of the peptide conjugate according to the invention, the protein ligand is glucagon.

In embodiments of the peptide conjugate according to the invention, the protein ligand is GLP-1.

In embodiments of the peptide conjugate according to the invention the protein ligand is a pharmacophore.

In embodiments the protein ligand is an agonist, antagonist and/or co-agonist of at least one class B1 G protein-coupled receptor. In embodiments said agonist, antagonist and/or co-agonist is tirzepatide. In embodiments said agonist, antagonist and/or co-agonist is LY3437943. In embodiments said agonist, antagonist and/or co-agonist is GIP. In said agonist, antagonist and/or co-agonist is glucagon. In embodiments said agonist, antagonist and/or co-agonist is GLP-1.

In embodiments, the amino acid sequence of the protein ligand of at least one class B1 G protein-coupled receptor, comprised within the peptide of the conjugate, comprises amino acid sequence variations, such as chemical modifications, deletions, substitutions and/or insertions of one or more amino acids in comparison to the naturally/endogenously (preferably in human) occurring amino acid sequence of the protein ligand of at least one class B1 G protein-coupled receptor.

In embodiments, the substitutions and/or insertions of one or more amino acids, comprise the substitution of amino acid(s) with and/or the insertion of one or more of
proteinogenic amino acids, and/or
non-proteinogenic amino acids, and/or
naturally occurring, and/or
artificial amino acids.

In embodiments of the peptide conjugate according to the invention, the amino acid sequence of the protein ligand of at least one class B1 G protein-coupled receptor, comprises at least one non-proteinogenic amino acid, preferably 2-aminoisobutyric acid.

In embodiments the at least one non-proteinogenic amino acid is or comprises 2-aminoisobutyric acid.

In embodiments of the peptide conjugate according to the invention, the protein ligand, which is preferably an agonist, antagonist and/or a co-agonist of at least one class B1 G protein-coupled receptor, is tirzepatide. In embodiments, and the tirzepatide does not comprise a conjugated fatty diacid module. In other words, the amino acid sequence of tirzepatide, which is comprised within the peptide of the conjugate, preferably does not comprise a conjugated fatty diacid module/molecule/side chain.

In embodiments of the peptide conjugate according to the invention, the protein ligand is an agonist, antagonist and/or co-agonist (of the at least one class B1 G protein-coupled receptor) that is GIP or tirzepatide, and the tirzepatide or GIP amino acid sequence comprises at least one non-proteinogenic amino acid, preferably 2-aminoisobutyric acid, and/or the tirzepatide does not comprise a conjugated fatty diacid module.

Examples of the aforementioned embodiments are conjugates comprising SEQ ID NOs 1-4, which comprise a partial sequence of either GIP (SEQ ID NOs 1-2 or 8-12) or Tirzepatide (SEQ ID NO 3-4 or 5-6 or 13) or of LY3437943 (SEQ ID NO 7 and 14), and wherein the amino acid sequence of the protein ligand, which is preferably an agonist, antagonist and/or a co-agonist of at least one class B1 G protein-coupled receptor, within the respective conjugate comprises at least one amino acid substitution and/or at least one non-proteinogenic amino acid, namely 2-aminoisobutyric acid.

In embodiments of the peptide conjugate according to the invention, the label is selected from the group comprising a fluorescent label, a nucleic acid label, a peptide label, an antibody or an antigen-binding fragment thereof, a biotin label, a chromogenic label, an MRI label, a metal label, or a radioactive label.

In embodiments of the peptide conjugate according to the invention, the label is an antibody or an antigen-binding fragment thereof.

In embodiments of the peptide conjugate according to the invention, the label is a chromogenic label. A chromogenic label preferably comprises an enzyme, such as peroxidases, e.g., horseradish peroxidase (HRP), alkaline phosphatase (AP), β-galactosidase or urease.

In embodiments of the peptide conjugate according to the invention, the label is a fluorescent label.

In embodiments of the peptide conjugate according to the invention, the fluorescent label is selected from the group consisting of cyanines, azetidine-substituted fluorescent compounds, such as Janelia Fluors, or rhodamines.

Non-limiting examples of cyanines are Cy3 (Cyanine-3), Cy5 (Cyanine-5) and Cy7 (Cyanine-7). Non-limiting examples of rhodamines are TMR, SIR, TMR-d12 and SiR-d12.

Non-limiting examples of azetidine-substituted fluorescent compounds are Janelia Fluors. Non-limiting examples of Janelia Fluors are Janelia Fluor549, Janelia Fluor646, Janelia Fluor585, Janelia Fluor635, Janelia Fluor669.

In embodiments, the Janelia Fluors are present as Janelia Fluor549-NHS Ester, Janelia Fluor646-NHS Ester, Janelia Fluor585-NHS Ester, Janelia Fluor635-NHS-Ester and Janelia Fluor669-NHS Ester.

Examples of fluorescent labels according to embodiments of the invention include, without being limited to, rhodamine and derivatives, lissamine, fluorescein, 5-bromomethylfluorescein and derivatives, DAPI, Hoechst 33258, R-phycocyanin, B-phycoerythrin, R-phycoerythrin, Lucifer Yellow, IAEDANS, 7-Me2N-coumarin-4-acetate, 7-OH-4-CH3-coumarin-3-acetate, monobromobiman, Pyrene trisulfonates such as Cascade Blue and monobromotrimethyl ammoniobiman, Texas Red, Rhodamine Green, Oregon Green 30 488, Oregon Green 514, 7-NH2-4CH3-25-coumarin-3-acetate (AMCA), FAM, TET, CAL Fluor Gold 540, JOE, VIC, Quasar 570, CAL Fluor Orange 560, NED, Oyster 556, TMR, CAL Fluor Red 590, HEX, ROX, LC Red 610, CAL Fluor Red 610, Texas Red, LC Red 610, CAL Fluor Red 610, LC Red 640, CAL Fluor Red 635, LC Red 670, Quasar 670, Oyster 645, LC Red 705, BODIPY FL, Cal Gold, BODIPY R6Gj, Yakima Yellow, Cal Orange, BODIPY TMR-X, JOE, HEX, Quasar-570, TAMRA, Rhodamine Red-X, Redmond Red, BODIPY 581/591, Cy3, Cy3.5, Cy5, Cy5.5, Cal Red/Texas Red, BODIPY 630/665-X, BODIPY TR-X, Quasar-670/Cy5, Pulsar-650, Dy590, Dy490, Dy636, Dy682, Atto-488, Atto-532, Atto-Rho-6G, Atto-Rho101, Atto-647N, Atto-680, BMN-488, BMN-505, BMN-536, BMN-562. In the context of embodiments of the present invention also indirect labels may be used as labels, such as chromogenic labels, including various enzymes well-known in the art, such as horseradish peroxidase (HRP), alkaline phosphatase (AP), β-galactosidase, urease, and the like.

In embodiments of the peptide conjugate according to the invention, the peptide (-portion) of the conjugate comprises a cysteine residue at its C-terminal end. In embodiments of the peptide conjugate according to the invention, the C-terminus of the peptide conjugate is amidated. In embodiments of the peptide conjugate according to the invention, the C-terminus of the peptide (-portion) of the conjugate is amidated.

In embodiments of the peptide conjugate according to the invention, the peptide of the conjugate comprises a cysteine residue at its C-terminal end and/or wherein the C-terminus of the peptide (-portion) of the conjugate is amidated. In embodiments of the peptide conjugate according to the invention, the peptide of the conjugate comprises a cysteine residue at its C-terminal end and/or wherein the C-terminus of the peptide conjugate is amidated.

In embodiments, the C-terminal cysteine residue is added to the peptide of the conjugate and/or is not naturally comprised within the (preferably human) natural/endogenous amino acid sequence of the protein ligand of at least one class B1 G protein-coupled receptor.

In embodiments, the peptide conjugate is characterized in that the amino acid sequence of the protein ligand of at least one class B1 G protein-coupled receptor, comprises one or more amino acid sequence variations, compared to a (preferably human) natural/endogenous amino acid sequence of the protein ligand, wherein the amino acid sequence variation comprises one or more chemical modifications, deletions, substitutions and/or insertions, wherein said substitutions and/or insertions are selected from the group comprising proteinogenic amino acids, non-proteinogenic amino acids, naturally occurring amino acids, synthetic amino acids and artificial amino acids.

The selection of the naturally/endogenously occurring amino acid sequence of the protein ligand is preferably dependent on the receptor(s) to be detected and/or labelled. In embodiments a naturally/endogenously occurring amino acid sequence is an amino acid sequence naturally/endogenously occurring in one or more mammalian species. In preferred embodiments the naturally/endogenously occurring amino acids sequence is an amino acid sequence that naturally/endogenously occurs in human.

In embodiments the at least one class B1 G protein-coupled receptor is a naturally/endogenously occurring protein in one or more mammalian species. In preferred embodiments the at least one class B1 G protein-coupled receptor is a naturally/endogenously occurring protein in human.

In embodiments, one or more additional consecutive amino acids are added between at the C-terminal end of the peptide (sequence/portion) of the conjugate and the C-terminal cysteine residue, preferably such that the cysteine residue is rendered more accessible for chemical and/or linking reactions. In embodiments said additional consecutive amino acids are selected from Glycine, Alanine, or any other suitable naturally occurring or synthetic amino acid.

In embodiments, said additional consecutive amino acids are or comprise at least one Glycine (Gly), preferably at least two Glycines.

An example of the aforementioned embodiments, conjugates comprising SEQ ID NOs 1-2 are presented, wherein the peptide of the conjugate comprises a partial amino acid sequence of natural/endogenous GIP (preferably human) and a C-terminal cysteine residue separated from the C-terminal end of the peptide (-portion) of the conjugate by two consecutive Glycines (Gly).

Accordingly, in embodiments, the amino acid sequence of the protein ligand of at least one class B1 G protein-coupled receptor, comprised within the present peptide conjugate can comprise amino acid substitutions, additions and/or deletions, such that said amino acid sequence shares at least 70% identity with the amino acid sequence of the naturally/endogenously (preferably in human) occurring protein ligand of the at least one class B1 G protein-coupled receptor. Greater levels of sequence identity are also envisaged, as disclosed herein.

In embodiments, where the protein ligand of at least one class B1 G protein-coupled receptor, is a chemical compound, pharmaceutical compound and/or drug, the term “naturally/endogenously occurring” refers to the amino acid sequence of said chemical compound, pharmaceutical compound and/or drug as it is provided by the supplier/manufacturer and/or as approved for therapeutic and/or research use. A non-limiting example of the “the naturally/endogenously occurring” amino acid sequence of tirzepatide would be the amino acid sequence of tirzepatide as provided by Eli Lilly (Eli Lilly and Company, USA).

It is further advantageous to incorporate non-proteinogenic and helix-inducing amino acids, such as 2-aminoisobutyric acid (Aib), into the amino acid sequence of the present peptide conjugate, allowing resistance against endopeptidases and hence longer peptide conjugate half-life for in vivo studies.

In embodiments of the peptide conjugate according to the invention, the peptide conjugate comprises a linker between the peptide (-portion) of the conjugate and the label, preferably a peptide linker.

In embodiments of the peptide conjugate according to the invention, the peptide of the conjugate comprises or consist of an amino acid sequence according to any one of SEQ ID NO 5-20, or a sequence of at least 70% identity thereto.

In embodiments of the peptide conjugate according to the invention, the amino acid sequence of the peptide conjugate comprises or consist of an amino acid sequence according to any one of SEQ ID NO 1-4, or a sequence of at least 70% identity thereto.

In embodiments of the peptide conjugate according to the invention, the amino acid sequence of the peptide conjugate comprises an amino acid sequence according to any one of SEQ ID NO 1-20, or a sequence of at least 70% identity thereto.

In another aspect the present invention relates to an in vitro method for assembling the peptide conjugate according to the invention, comprising covalently linking the peptide of the conjugate to the label, wherein the linking rection is preferably a click reaction, and wherein the peptide of the conjugate comprises a cysteine residue at its C-terminal end.

In embodiments of the in vitro method for assembling the peptide conjugate according to the invention the peptide conjugate is synthesized in vitro, e.g., on solid phase support.

In embodiments of the in vitro method for assembling the peptide conjugate according to the invention the peptide conjugate is subjected to global deprotection after its synthesis.

In embodiments of the in vitro method for assembling the peptide conjugate according to the invention the peptide conjugate is purified by reverse-phase High Performance Liquid Chromatography (HPLC) after its synthesis.

In embodiments of the in vitro method for assembling the peptide conjugate according to the invention the peptide conjugate is subjected to a characterization step by liquid chromatography-mass spectrometry (LC-MS) and/or high-resolution mass spectrometry (HRMS) after its synthesis.

In specific embodiments the in vitro method for assembling the peptide conjugate according to the invention comprises covalently linking at least one peptide (-portion) of the conjugate to a label. In some embodiments the label is a fluorescent label, as described herein. In other embodiments the label is a label as described herein.

In embodiments the step of covalent linking is achieved using a “click chemistry” reaction.

In one embodiment a click chemistry reaction or click reaction takes advantage of or comprises the functionalization of the peptide (-portion) of the present conjugate with an N-terminal cysteine, such that the functionalized N-terminus of the peptide can be covalently linked to a peptide linker or a label with said click reaction.

In embodiments the click chemistry reaction is achieved or allowed to occur during an incubation period, e.g., of at least 5 minutes, preferably of an incubation period between 5 minutes and 48 hours. In some embodiments the incubation is performed at room temperature (RT). The incubation time and temperature may vary depending on the click reaction to be performed. A skilled person knows how to adjust the reaction and incubation parameters of a respective click chemistry reaction.

Common examples for click chemistry or click (chemistry) reactions are pericyclic reactions between azides and (strained) alkynes, between tetrazines and strained double or triple bonds, or between thiols and maleimides, described by a so-called non-pericyclic Michael reaction.

Herein, conjugating preferably means covalently linking and said terms may be used interchangeably in preferred embodiments.

In embodiments the in vitro method for assembling the peptide conjugate according to the invention, comprises the steps of
a) in vitro synthesizing at least one peptide, preferably on solid phase support,
b) optionally subjecting the at least one peptide obtained in step (a) to global deprotection,
c) optionally purifying the at least one peptide obtained in step (a) or (b), preferably by reverse-phase HPLC, and/or
d) optionally characterizing the at least one peptide obtained in step (a), (b) or (c) by LC-MS and/or HRMS,
e) optionally covalently linking the at least one peptide obtained in step (a), (b), (c) or (d) to a label and/or at least one further peptide, optionally obtained according to any one of steps (a)-(d) or any combination thereof.

The person skilled in the art is aware of means and methods suitable for synthesizing and conjugating peptides in vitro.

In a further aspect, the present invention relates to an in vitro method for labelling and/or detecting the presence and/or localization of class B1 G protein-coupled receptors in a sample, comprising
contacting a sample with a peptide conjugate according to the invention, wherein the sample comprises at least one cell, wherein the peptide conjugate binds to a class B1 G protein-coupled receptor present within the cell and/or the cell membrane of the at least one cell, and
detecting the presence and/or localization of the peptide conjugate bound to a class B1 G protein-coupled receptor in the at least one cell and/or the sample.

In embodiments the sample is a cell culture or patient sample. In embodiments the sample is a tissue sample of a patient.

In embodiments the label of the peptide conjugate is a fluorescent label and detecting the presence and/or localization of the peptide conjugate bound to a class B1 G protein-coupled receptor in at least one cell and/or the sample comprises fluorescence imaging, such as fluorescence microscopy, single molecule localization microscopy, nanoscopy, fluorescence activated cell sorting (FACS) or spectroscopy.

Embodiments of the present peptide conjugates can be used for immunohistochemistry detection of a B1 G protein-coupled receptor in a sample, such as a tissue sample. In embodiments the present peptide conjugates can be used for immunohistochemistry detection, wherein the label of the peptide conjugate is preferably a fluorescent label or a chromogenic label. A chromogenic label preferably comprises an enzyme, such as peroxidases, e.g., horseradish peroxidase (HRP), alkaline phosphatase (AP), β-galactosidase or urease.

In embodiments of the in vitro method, the label of the peptide conjugate is a chromogenic label and detecting the presence and/or localization of the peptide conjugate bound to a class B1 G protein-coupled receptor in at least one cell and/or the sample comprises contacting the sample with a substrate of the enzyme, which is comprised within the chromogenic label, and detecting the signal generated upon processing of the substrate by the enzyme. In embodiments, the presence of a peptide conjugate bound to a class B1 G protein-coupled receptor in a sample, results during detection in the processing of the substrate by the enzyme comprised within the chromogenic label, which results in a detectable signal, such as a change in color or the emission of light.

The person skilled in the art is familiar with suitable methods for immunohistochemistry staining and for detecting a chromogenic label.

In embodiments the label of the peptide conjugate is a radioactive label and detecting the presence and/or localization of the peptide conjugate bound to a class B1 G protein-coupled receptor in the at least one cell and/or the sample comprises X-ray detection.

The person skilled in the art is familiar with suitable methods for X-ray detection.

In embodiments the label of the peptide conjugate is a magnetic, metal, iodine, barium sulfate and/or MRI label and detecting the presence and/or localization of the peptide conjugate bound to a class B1 G protein-coupled receptor in at least one cell and/or the sample comprises magnetic resonance imaging (MRI), Positron emission tomography-magnetic resonance imaging (PET-MRI), computed tomography (CT) or fluoroscopy.

The person skilled in the art is familiar with suitable methods for detecting a magnetic, iodine, barium sulfate, metal and/or MRI label.

In embodiments the label of the peptide conjugate is an oligonucleotide and detecting the presence and/or localization of the peptide conjugate bound to a class B1 G protein-coupled receptor in at least one cell and/or the sample comprises next generation sequencing (NGS) or spatial transcriptomics.

The person skilled in the art is familiar with suitable methods for detecting an oligonucleotide or spatial transcriptomics.

In embodiments the present peptide conjugates can be used to label and detect B1 G protein-coupled receptors in a sample. In embodiments the present peptide conjugates can be used to label and detect the localization, protein interactions, lifetime and/or expression of B1 G protein-coupled receptors on a molecular level, e.g., on a cellular level and/or in tissues.

As suitable antibodies targeting B1 G protein-coupled receptors are still lacking, little is known yet about the cellular and tissue localization of B1 G protein-coupled receptors and their role in molecular mechanisms, e.g., in a healthy physiological context and in disease. The present peptide conjugates constitute a useful tool to overcome this lack of research tools and the resulting lack of knowledge and can serve to unravel cellular and tissue localization, expression and functions of B1 G protein-coupled receptors. Accordingly, embodiments of the present invention are particularly useful for research on type 2 diabetes and related molecular mechanisms.

In another aspect, the present invention comprises the use of the peptide conjugate according to the invention for labelling class B1 G protein-coupled receptors in vitro.

In another aspect, the present invention comprises the use of the peptide conjugate according to the invention for labelling class B1 G protein-coupled receptors in vivo.

In another aspect, the invention relates to a nucleic acid molecule encoding either the peptide conjugate according to the invention or a fragment thereof, wherein the fragment is composed of the peptide and optionally a peptide linker sequence, and wherein the peptide comprises an amino acid sequence of GLP-1, GIP, glucagon or a precursor protein thereof, or an amino acid sequence with at least 70% identity thereto.

Embodiments of a peptide of the conjugate comprising an amino acid sequence
of GLP-1, GIP, glucagon or a precursor protein thereof, or
of dual and or triple agonists of class B1 G protein-coupled receptors comprising sequences comprised of combinations of GLP-1, GIP and glucagon, or combinations thereof, comprise, for example, sequence variants that may exhibit a sequence identity of 45, 50, 55, 60, 65, 70, 75, 80, 81, 82, 83, 84, 85, 86, 87, 88, 89, 90, 91, 92, 93, 94, 95, 96, 97, 98 or 99%, or a sequence identity of at least 70% or higher, to a, preferably mammalian natural/endogenous, amino acid sequence of GLP-1, GIP, glucagon or a precursor protein thereof, or of dual and or triple agonists of class B1 G protein-coupled receptors comprising sequences comprised of combinations of GLP-1, GIP and glucagon, or combinations thereof.

Sequence identity may be determined using methods known to one skilled in the art, such as BLAST or other sequence alignment tools.

In embodiments the nucleic acid molecule according to the invention comprises one of the nucleic acid sequences SEQ ID NO 21-25, or part thereof, or a sequence at least 70% identical thereto and/or encodes an amino acid sequence comprising one of SEQ ID NO 1-20 or a sequence at least 70% identical thereto.

In another aspect the invention relates to a Kit comprising:
the peptide conjugate according to the invention, and/or
a nucleic acid according to the invention, and
optionally a linker, and/or
optionally a label, and/or
optionally means for performing a linking reaction, preferably a click-reaction.

In embodiments, the kit comprises:
the peptide conjugate as described herein,
   or
a peptide and a label, wherein the peptide comprises an amino acid sequence of a protein ligand of at least one class B1 G protein-coupled receptor, or comprises an amino acid sequence with least 70% identity to said protein ligand, wherein the peptide and the label are configured for covalent linkage,
optionally a linker, and/or
optionally means for performing a linking reaction, preferably a click-reaction.

The present disclosure further includes kits and packages containing the herein described peptide conjugates.

TABLE 1
Amino acid sequences comprised within or constituting exemplary embodiments of the
peptide conjugate according to the invention. Bold typeface stands for non-
proteinogenic amino acids, underlined typeface stands for insertions, italic typeface
illustrates substitutions compared to the natural/endogenously occurring amino acid
sequence of the respective protein ligand, which is preferably an agonist, antagonist
and/or a co-agonist of at least one class B1 G protein-coupled receptor. “-NH₂” stands
for C-terminal amidation, “H-” refers to the N-terminus (NH₂-terminus) of the amino
acid sequence. “Aib” stands for 2-aminoisobutyric acid, ″αMeL13” stands for alpha-
methyl-L-leucine. “(Cy3)” or “(Cy5)” refer to the respective fluorophore (label),
optionally further comprising a linker, e.g., peptide linker, sequence (not shown here).

| SEQ ID | | |
|---|---|---|
| NO | Name | Amino acid sequence |
| 1 | sGIP549 | H- |
| | | YAibEGTFISDYSIAMDKIRQQDFVNWLLAQRGKKSDWKHNITQGG |
| | | C(Cy3)-NH₂ |
| 2 | sGIP648 | H- |
| | | YAibEGTFISDYSIAMDKIRQQDFVNWLLAQRGKKSDWKHNITQGG |
| | | C(Cy5)-NH₂ |
| 3 | Tirzepatide-Cy3 | H- |
| | | YAibEGTFTSDYSIAibLDKIAQKAFVQWLIAGGPSSGAPPPC(Cy3)- |
| | | NH₂ |
| 4 | Tirzepatide-Cy5 | H- |
| | | YAibEGTFTSDYSIAibLDKIAQKAFVQWLIAGGPSSGAPPPC(Cy5)- |
| | | NH₂ |
| 5 | Tirzepatide (full- | H-YAibEGTFTSDYSIAibLDKIAQKAFVQWLIAGGPSSGAPPPS-NH₂ |
| | length sequence) | |
| 6 | Tirzepatide-partial | H-YAibEGTFTSDYSIAibLDKIAQKAFVQWLIAGGPSSGAPPPC-NH₂ |
| | sequence | |
| 7 | LY3437943 (triple | H- |
| | glucagon, GIP and | YAibQGTFTSDYSIαMeL13LDKKAQAibAFIEYLLEGGPSSGAPPPS- |
| | GLP1 receptor | NH₂ |
| | agonist) | |
| 8 | GIP-partial | H- |
| | sequence | YAibEGTFISDYSIAMDKIRQQDFVNWLLAQRGKKSDWKHNITQGG |
| | | C-NH₂ |
| 9 | GIP-partial | YAibEGTFISDYSIAMDKIRQQDFVNWLLAQRGKKSDWKHNITQGG |
| | sequence V2 | C |
| 10 | GIP partial | YAibEGTFISDYSIAMDKIRQQDFVNWLLAQRGKKSDWKHNITQ |
| | sequence V3 | |
| 11 | GIP partial | H-YAEGTFISDYSIAMDKIRQQDFVNWLLAQRGKKSDWKHNITQ- |
| | sequence V4 | NH₂ |
| 12 | GIP partial | YAEGTFISDYSIAMDKIHQQDFVNWLLAQKGKKNDWKHNITQ |
| | sequence V5 | |
| 13 | Tirzepatide-partial | YAibEGTFTSDYSIAibLDKIAQKAFVQWLIAGGPSSGAPPP |
| | sequence V2 | |
| 14 | LY3437943 | YAibQGTFTSDYSIαMeL13LDKKAQAibAFIEYLLEGGPSSGAPPPS |
| | sequence V2 | |
| 15 | GIP wt human (153 | MVATKTFALLLLSLFLAVGLGEKKEGHFSALPSLPVGSHAKVSSPQ |
| | aa; NP_004114.1) | PRGPRYAEGTFISDYSIAMDKIHQQDFVNWLLAQKGKKNDWKHNIT |
| | | QREARALELASQANRKEEEAVEPQSSPAKNPSDEDLLRDLLIQELL |
| | | ACLLDQTNLCRLRSR |
| 16 | Pro-glucagon, | MKSIYFVAGLFVMLVQGSWQRSLQDTEEKSRSFSASQADPLSDPD |
| | GLUC_HUMAN | QMNEDKRHSQGTFTSDYSKYLDSRRAQDFVQWLMNTKRNRNNIA |
| | (180 aa; | KRHDEFERHAEGTFTSDVSSYLEGQAAKEFIAWLVKGRGRRDFPE |
| | NP_002045.1) | EVAIVEELGRRHADGSFSDEMNTILDNLAARDFINWLIQTKITDRK |
| 17 | Glucagon (29 aa; | HSQGTFTSDYSKYLDSRRAQDFVQWLMNT |
| | GCG) | |
| 18 | Glucagon-like | HDEFERHAEGTFTSDVSSYLEGQAAKEFIAWLVKGRG |
| | peptide-1 (37 aa; | |
| | GLP) | |
| 19 | Glucagon-like | HAEGTFTSDVSSYLEGQAAKEFIAWLVKGR |
| | peptide-1(aa 7-36 | |
| | of GLP-1; length 30 | |
| | aa) | |
| 20 | Glucagon-like | HAEGTFTSDVSSYLEGQAAKEFIAWLVKGRG |
| | peptide 1 (aa 7-37 | |
| | of GLP-1; length 31 | |
| | aa) | |
| 21 | Homo sapiens | AGCAGGCTCAGAAGGTCCAGAAATCAGGGGAAGGAGACCCCTA |
| | gastric inhibitory | TCTGTCCTTCTTCTGGAAGAGCTGGAAAGGAAGTCTGCTCAGGA |
| | polypeptide (GIP), | AATAACCTTGGAAGATGGTGGCCACGAAGACCTTTGCTCTGCTG |
| | cDNA | CTGCTGTCCCTGTTCCTGGCAGTGGGACTAGGAGAGAAGAAAG |
| | (NM_004123.3) | AGGGTCACTTCAGCGCTCTCCCCTCCCTGCCTGTTGGATCTCAT |
| | | GCTAAGGTGAGCAGCCCTCAACCTCGAGGCCCCAGGTACGCG |
| | | GAAGGGACTTTCATCAGTGACTACAGTATTGCCATGGACAAGAT |
| | | TCACCAACAAGACTTTGTGAACTGGCTGCTGGCCCAAAAGGGG |
| | | AAGAAGAATGACTGGAAACACAACATCACCCAGAGGGAGGCTC |
| | | GGGCGCTGGAGCTGGCCAGTCAAGCTAATAGGAAGGAGGAGG |
| | | AGGCAGTGGAGCCACAGAGCTCCCCAGCCAAGAACCCCAGCG |
| | | ATGAAGATTTGCTGCGGGACTTGCTGATTCAAGAGCTGTTGGCC |
| | | TGCTTGCTGGATCAGACAAACCTCTGCAGGCTCAGGTCTCGGT |
| | | GACTCTGACCACACCCAGCTCAGGACTGGATTCTGCCCTTCACT |
| | | TAGCACCTGCCTCAGCCCCACTCCAGAATAGCCAAGAGAACCC |
| | | AAACCAATAAAGTTTATGCTAAGTCGAGCCCATTGTGAAAATTTA |
| | | TTAAAAT GACTACTGAGCACTAA |
| 22 | Homo sapiens | ACAGAGCTTAGGACACAGAGCACATCAAAAGTTCCCAAAGAGG |
| | glucagon (GCG), | GCTTGCTCTCTCTTCACCTGCTCTGTTCTACAGCACACTACCAG |
| | cDNA | AAGACAGCAGAAATGAAAAGCATTTACTTTGTGGCTGGATTATTT |
| | (NM_002054.5)- | GTAATGCTGGTACAAGGCAGCTGGCAACGTTCCCTTCAAGACAC |
| | The protein | AGAGGAGAAATCCAGATCATTCTCAGCTTCCCAGGCAGACCCAC |
| | encoded by this | TCAGTGATCCTGATCAGATGAACGAGGACAAGCGCCATTCACAG |
| | gene is a pre-pro- | GGCACATTCACCAGTGACTACAGCAAGTATCTGGACTCCAGGC |
| | protein that is | GTGCCCAAGATTTTGTGCAGTGGTTGATGAATACCAAGAGGAAC |
| | cleaved into GLP-1, | AGGAATAACATTGCCAAACGTCACGATGAATTTGAGAGACATGC |
| | GLP-2, GCG and | TGAAGGGACCTTTACCAGTGATGTAAGTTCTTATTTGGAAGGCC |
| | glicentin-related | AAGCTGCCAAGGAATTCATTGCTTGGCTGGTGAAAGGCCGAGG |
| | polypeptide | AAGGCGAGATTTCCCAGAAGAGGTCGCCATTGTTGAAGAACTTG |
| | (GRPP) mature | GCCGCAGACATGCTGATGGTTCTTTCTCTGATGAGATGAACACC |
| | peptides. | ATTCTTGATAATCTTGCCGCCAGGGACTTTATAAACTGGTTGATT |
| | | CAGACCAAAATCACTGACAGGAAATAACTATATCACTATTCAAGA |
| | | TCATCTTCACAACATCACCTGCTAGCCACGTGGGATGTTTGAAA |
| | | TGTTAAGTCCTGTAAATTTAAGAGGTGTATTCTGAGGCCACATTG |
| | | CTTTGCATGCCAATAAATAAATTTTCTTTTAGTGTTGTGTAGCCA |
| | | AAAATTACAAATGGAATAAAGTTTTATCAAAATATTGCTAAAATAT |
| | | CAGCTTTAAAATATGAAAGTGCTAGATTCTGTTATTTTCTTCTTAT |
| | | TTTGGATGAAGTACCCCAACCTGTTTACATTTAGCGATAAAATTA |
| | | TTTTTCTATGATATAATTTGTAAATGTAAATTATTCCGATCTGACA |
| | | TATCTGCATTATAATAATAGGAGAATAGAAGAACTGGTAGCCACA |
| | | GTGGTGAAATTGGAAAGAGAACTTTCTTCCTGAAACCTTTGTCTT |
| | | AAAAATACTCAGCTTTCAATGTATCAAAGATACAATTAAATAAAAT |
| | | TTTCAAGCTTC |
| 23 | Pro-glucagon cDNA | ATGAAAAGCATTTACTTTGTGGCTGGATTATTTGTAATGCTGGTA |
| | | CAAGGCAGCTGGCAACGTTCCCTTCAAGACACAGAGGAGAAAT |
| | | CCAGATCATTCTCAGCTTCCCAGGCAGACCCACTCAGTGATCCT |
| | | GATCAGATGAACGAGGACAAGCGCCATTCACAGGGCACATTCA |
| | | CCAGTGACTACAGCAAGTATCTGGACTCCAGGCGTGCCCAAGA |
| | | TTTTGTGCAGTGGTTGATGAATACCAAGAGGAACAGGAATAACA |
| | | TTGCCAAACGTCACGATGAATTTGAGAGACATGCTGAAGGGACC |
| | | TTTACCAGTGATGTAAGTTCTTATTTGGAAGGCCAAGCTGCCAA |
| | | GGAATTCATTGCTTGGCTGGTGAAAGGCCGAGGAAGGCGAGAT |
| | | TTCCCAGAAGAGGTCGCCATTGTTGAAGAACTTGGCCGCAGAC |
| | | ATGCTGATGGTTCTTTCTCTGATGAGATGAACACCATTCTTGATA |
| | | ATCTTGCCGCCAGGGACTTTATAAACTGGTTGATTCAGACCAAA |
| | | ATCACTGACAGGAAA |
| 24 | Glucagon | CATTCACAGGGCACATTCACCAGTGACTACAGCAAGTATCTGGA |
| | cDNA | CTCCAGGCGTGCCCAAGATTTTGTGCAGTGGTTGATGAATACC |
| 25 | Glucagon-like | CACGATGAATTTGAGAGACATGCTGAAGGGACCTTTACCAGTGA |
| | peptide-1 | TGTAAGTTCTTATTTGGAAGGCCAAGCTGCCAAGGAATTCATTG |
| | cDNA | CTTGGCTGGTGAAAGGCCGA |

In embodiments, the peptide conjugate or the peptide (-portion) of the conjugate according to the invention is also envisaged to consist of or comprise the amino acid sequences disclosed in table 1, with or without (chemical) modifications, e.g., of amino acid side chains of the above-disclosed amino acid sequences, and/or with or without terminal (chemical) modifications, e.g., of N- or C-termini of the above-disclosed amino acid sequences.

Embodiments and features of the invention described with respect to the peptide conjugates, the method and kits are considered to be disclosed with respect to each and every other aspect of the disclosure, such that features characterizing the peptide conjugates, may be employed to characterize the methods, or kit and vice-versa. The various aspects of the invention are unified by, benefit from, are based on and/or are linked by the common and surprising finding of the successful labelling and detection of class B1 G protein-coupled receptors with the peptide conjugates described herein.

### DETAILED DESCRIPTION OF THE INVENTION

All cited documents of the patent and non-patent literature are hereby incorporated by reference in their entirety.

The present invention is directed to a peptide conjugate comprising a peptide covalently linked to a label, wherein the peptide comprises an amino acid sequence of a protein ligand of at least one class B1 G protein-coupled receptor, which is preferably an agonist, antagonist and/or a co-agonist of at least one class B1 G protein-coupled receptor, or comprises a sequence with least 70% identity to said protein ligand of at least one class B1 G protein-coupled receptor.

In embodiments of the peptide conjugate according to the invention, the at least one class B1 G protein-coupled receptor is selected from the Glucagon-like subfamily comprising Glucagon-like peptide 1 receptor (GLP-1R), glucose-dependent insulinotropic polypeptide receptor (GIPR), GLP-2R, GCGR, GHRHR and SCTR. In embodiments of the peptide conjugate according to the invention, the protein ligand of at least one class B1 G protein-coupled receptor, is selected from the group consisting of GLP-1, GIP, glucagon or a precursor protein thereof, or dual and triple agonists of class B1 G protein-coupled receptors comprising sequences of GLP-1, GIP or glucagon or combinations thereof. Examples of dual and triple agonists are tirzepatide and LY3437943.

“G protein-coupled receptors” are the most abundant membrane proteins involved in numerous physiological functions, wherein one G protein-coupled receptors class is the “class B1 family receptors”, which comprises 15 receptors for peptide hormones that regulate numerous biological processes, such as development, growth, metabolism and neurological activity. “Class B1 G protein-coupled receptors” are important therapeutic targets for several diseases such as analogues of the receptor agonist of glucagon-like peptide 1 (GLP-1) and Glucose-dependent insulinotropic polypeptide (GIP). Herein B1 G protein-coupled receptors refer to receptors selected from the Glucagon-like subfamily comprising Glucagon-like peptide 1 receptor (GLP-1R), glucose-dependent insulinotropic polypeptide receptor (GIPR), GLP-2R, GCGR and SCTR.

“Pro-glucagon” (see SEQ ID NO. 16) is post-translationally processed in a tissue-specific manner in pancreatic A cells and intestinal L cells. In pancreatic A cells, the major bioactive hormone is glucagon (see SEQ ID NO. 17), which is cleaved by PCSK2/PC2 and comprises the amino acid sequence of aa 92-128 of pro-glucagon. In the intestinal L cells PCSK1/PC1 cleaves GLP-1 (see SEQ ID NO. 18), GLP-2, glicentin and oxyntomodulin. GLP-1 comprises the amino acid sequence of aa 92-128 of pro-glucagon and is further N-terminally truncated by post-translational processing in the intestinal L cells resulting in GLP-1 (7-37) GLP-1-(7-36) (see SEQ ID No. 19 and 20). In vivo GLP-1 (7-36) is C-terminally amidated, wherein said amidation is neither important for the metabolism of GLP-1 nor for its effects on the endocrine pancreas.

A post-translational modification (PTM) is a covalent processing event resulting from a proteolytic cleavage or from the addition of a modifying group to one amino acid. PTMs modulate the function of proteins by altering their activity state, localization, turnover, and/or interactions with other proteins. Although proteins can be modified pre-, co- or post-translationally, all protein modifications are commonly referred to as PTMs, as a majority of them are made post-translationally, after the protein is folded.

“Glucagon-like peptide-1” (GLP-1) is a 30- or 31-amino-acid-long peptide hormone deriving from the tissue-specific posttranslational processing of the proglucagon peptide. It is produced and secreted by intestinal enteroendocrine L-cells and certain neurons within the nucleus of the solitary tract in the brainstem upon food consumption and nutrient stimulation. Upon binding to its receptors (GLP-1Rs) insulin is secreted and glucagon secretion inhibited. This leads to reduced gastric emptying rate and the stimulation of satiety via the central nervous system.

“Glucose-dependent insulinotropic polypeptide” (GIP), also known as Gastric inhibitory polypeptide or gastric inhibitory peptide or gastric inhibitory peptide, is a hormone of the secretin family that plays an important role in the stimulation of insulin secretion. The GIP gene is expressed in gastrointestinal K cells of the mucosa of the duodenum and the jejunum. The GIP hormone is derived from a 153-amino acid proprotein and circulates as biologically active 42-amino acid peptide.

GIP, along with glucagon-like peptide-1 (GLP-1), belongs to a class of molecules referred to as incretins. Upon nutrient ingestion the postprandial insulin secretion is mainly caused by an increase in plasma glucose levels and the nutrient-induced secretion of the gastrointestinal hormones GLP-1 and GIP. Sharing the essential physiological task of stimulating postprandial insulin secretion, GLP-1R and GIPR in beta cells have many common intracellular pathways.

“Glucagon” is a peptide hormone, produced by alpha cells of the pancreas. It raises concentration of glucose and fatty acids in the bloodstream and is considered the body's main catabolic hormone. Glucagon is produced from the 160-amino acid protein proglucagon, encoded by the GCG gene. The pancreas releases glucagon when the amount of glucose in the bloodstream is too low. Glucagon causes the liver to engage in glycogenolysis: converting stored glycogen into glucose, which is released into the bloodstream. Glucagon binds to the glucagon receptor (GCGR), a plasma membrane G protein-coupled receptor, wherein the conformational change in the receptor activates downstream G proteins.

“Glucose-dependent insulinotropic polypeptide receptor” (GIPR) is a class B1 G protein-coupled receptor. GIPR is expressed at the pancreatic beta cells and other tissues, such as the nervous system, the gastrointestinal tract and the cardiovascular system, in adipocytes and bone cells. GIP uses its C-terminal alpha-helix (position 6-30) to interact the N-terminal extracellular domain of the GIPR. This interaction results in the interaction of the N-terminus of GIP with the receptor core, whereupon the GIPR undergoes conformational changes and induces downstream intracellular signaling. Endogenous GIPR ligands include GIP (1-42), GIP (1-30) NH2, GIP (3-42), and GIP (3-30) NH2.

“Glucagon-like peptide 1 receptor” (GLP-1R) is a class B1 G protein-coupled receptor and consists of three domains, an extracellular N-terminus, a transmembrane core domain, and an intracellular C-terminal domain. The GLP-1R N-terminus binds to the C-terminus of the GLP-1 peptide while the transmembrane domain binds to the N-terminus of the GLP-1 peptide, leading to the activation of the receptor and downward signaling, e.g., by G proteins. GLP-1R is present in cells of the endocrine islets of the pancreas, the cardiovascular system, the gastrointestinal tract, brain, kidney, and immune cells.

“Glucagon-like peptide 1 receptor” (GLP-1R) and glucose-dependent insulinotropic polypeptide receptor (GIPR) are two class B1 G protein-coupled receptors. In beta cells of the pancreas the activation of both receptors leads to increased cyclic adenosine monophosphate (cAMP) and glucose-dependent insulin secretion. The GLP-1R-GIPR peptide-based co-agonist tirzepatide has been shown to improving glycemic control in type 2 diabetes patients. Tirzepatide's mechanism of action comprises GIPR signaling and biased GLP-1R signaling.

“Tirzepatide” (TZP or Tz) is a linear peptide containing 39 amino acids. Commonly the lysine residue at position 20 is conjugated to a C20 fatty diacid module via a linker, which achieves a long-term effect when Tirzepatide is administered to a patient. In embodiments of the present invention, however, tirzepatide preferably does not comprise a conjugated fatty diacid module. Amino acid sequence positions 2 and 13 of Tirzepatide contain two non-coding amino acid residues (Aib, α-aminoisobutyric acid), and the C-terminus is amidated.

The structure of Tirzepatide (TZP or Tz) is a combination of GIP partial peptide sequence analogs and Exenatide partial peptide sequence. Exenatide is a medication used to treat diabetes mellitus type 2, which binds to the intact human GLP-1R in a similar way to the human GLP-1, and wherein Exenatide bears a 50% amino acid homology to GLP-1 and has a longer half-life in vivo. Tirzepatide, also referred to as a “dual” receptor agonist, achieves synergistic effects as a GLP-1R-GIPR co-agonist, by promoting higher insulin responses than separate administration of each hormone. Currently other GLP-1R and/or GIPR agonists or dual or triple agonists, such as Dulaglutide, Lixisenatide, Exenatide, Semaglutide, Albiglutide, Liraglutide, Avexitide, HISHS-2001, DA-JC4, Peptide-19 or MAR709 and the GLP-1R/GIPR/GcgR tri-(triple-) agonist SAR441255/LY3437943 are in preclinical or clinical development. In some embodiments of the invention said agonists or co-agonists may be used instead or alternatively to tirzepatide.

“2-Aminoisobutyric acid” (also known as α-aminoisobutyric acid, AIB/Aib, α-methylalanine, or 2-methylalanine) is the non-proteinogenic amino acid with the structural formula H₂N—C(CH₃)₂—COOH. It is a strong helix inducer in peptides due to the Thorpe-Ingold effect of its gem-dimethyl group. The Thorpe-Ingold effect, or gem-dimethyl effect is an effect observed in chemistry where increasing steric hindrance favors ring closure and intramolecular reactions, e.g., 2-aminoisobutyric acid residues containing quaternary carbons are used to promote formation of certain types of helices. Oligomers of AIB form 310 helices. 2-Aminoisobutyric acid (Aib) is compatible with ribosomal elongation of peptide synthesis.

“LY3437943” (C₂₂₁H₃₄₂N₄₆O₆₈) is a triple agonist peptide of the glucagon receptor (GCGR), glucose dependent insulinotropic polypeptide receptor (GIPR), and glucagon-like peptide-1 receptor (GLP-1R).

A “peptide conjugate” or briefly “conjugate” refers in the context of the present invention to a peptide or amino acid sequence being linked or conjugated to another moiety, such as a label. The linking can be a direct covalent linkage to the other moiety, e.g., label, or can comprise a linking sequence or linker in between the amino acid sequence and the other moiety. In preferred embodiments the linker or linking sequence, if present, is an amino acid sequence or any other suitable linker to connect an amino acid sequence to a label. The person skilled in the art is aware of different techniques to link an amino acid sequence to another moiety, e.g., a label.

A “click reaction” (“click chemistry”) refers to a linking rection joining a substrate of choice with specific biomolecules, such as a biomolecule and a reporter molecule or label. Click reactions are defined by the use of mild reaction conditions (ambient temperature and pressure) in and compatible with water as a solvent. As such, they can be useful in the detection, localization and qualification of biomolecules. In chemical synthesis, click chemistry is a class of biocompatible small molecule reactions commonly used in bioconjugation. Click chemistry commonly is not limited to a single specific reaction but refers to a mode of generating products that follow examples in nature by joining small modular units together. However, click chemistry is not limited to biological conditions.

“Covalently linked” refers herein to the linkage of two molecules (e.g., an amino acid sequence and a label) through a covalent bond. A covalent bond is a chemical bond in which electrons are exchanged to form electron pairs between atoms, in chemistry it refers to the interatomic bond formed by the sharing of a pair of electrons between two atoms. This bond is formed by the electrostatic attraction of their nuclei to the same electrons. A covalent bond occurs when the bonded atoms have a lower total energy than that of widely separated atoms.

As used herein, a “ligand” is a substance that forms a complex with a biomolecule. In protein-ligand binding in natural systems, the ligand may produce a signal by binding to a site on a target protein. The binding typically, but must not necessarily, result in a change of conformational isomerism (conformation) of the target protein. Binding occurs by intermolecular forces, such as ionic bonds, hydrogen bonds and Van der Waals forces. The association or docking is typically reversible through dissociation. Optionally, ligand binding to a receptor protein may alter the conformation of the target by affecting the three-dimensional shape orientation. Ligands include, without limitation, agonists, antagonists and/or co-agonists of a receptor. The rate of binding between ligand and receptor target is typically referred to by affinity, and this measurement typifies a tendency or strength of the effect. Binding affinity is actualized not only by host-guest interactions, but also by solvent effects that can play a dominant, steric role which drives non-covalent binding in solution. The solvent provides a chemical environment for the ligand and receptor to adapt, and thus accept or reject each other as partners. Suitable methods and reaction conditions to determine specific ligand-receptor target interactions are known to a skilled person.

Herein an “agonist” is a chemical or compound, preferably a compound, peptide or protein, that activates a receptor and causes a downstream biological response. In preferred embodiments of the invention agonists are compounds, peptides or proteins that activate at least one class B1 G protein-coupled receptor. A physiological agonist is a substance that creates the same bodily responses, as a certain agonist, but does not bind to the same receptor. An endogenous agonist for a particular receptor is a compound naturally produced by the body that binds to and activates that receptor. A “dual agonist” preferably is able to bind to two different receptors, either with comparable or with different binding kinetics.

Herein a “co-agonist” is an agonist that cooperates with other co-agonists to jointly produce the desired effect. For example, some receptors are only activated if they are bound to two or more co-agonists at the same time. In embodiments a co-agonist may be a positive, negative, neutral or allosteric modulator of a receptor. Allosteric receptor modulators are substances that bind to a receptor to change the receptor's response to a stimulus. The site on the receptor to which an allosteric modulator binds to (e.g., an allosteric site) is different from the binding site of an endogenous agonist of the receptor. Accordingly, herein a protein ligand may be in embodiments an allosteric receptor modulator. Tirzepatide, is considered to be a “dual” GIP/GLP-1 receptor co-agonist, meaning that it is able to bind to both GIP and GLP-1 receptor. Herein, single, dual and triple co-agonists may also be comprised within the terms of “single, dual and triple agonists”. Hence, for example, tirzepatide may herein also be comprised within/referred to by the term “dual agonist”.

“Dual agonists” or “dual receptor agonists” or “dual (receptor) co-agonists” are considered to have respectively agonist and/or co-agonist activity at two different receptors, e.g., GLP-1R and GIPR. Due to synergistic action, the effect of dual agonists, or some dual co-agonists like tirzepatide, can be greater than that of individual single receptor agonists alone. Simultaneous activation of two receptors, preferably present in the same signaling network or pathway, may not only induce normal signal transduction but has also been shown to be beneficial by synergistically enhancing each other's effects, such as for dual (co-)agonists of GLP-1R and GIPR. In patients with type 2 diabetes, dual (co-)agonists, such as tirzepatide, have been recently shown to provide great benefit for both glycemic control and weight loss. “Triple agonists”, “Tri agonists” or “triple receptor agonists” are capable of binding to three different receptors. One example of a receptor triple agonist is LY3437943, which can bind as agonist to three different receptors, namely glucagon receptor, GIPR, and GLP-1R.

In contrast to agonists, an “antagonist” blocks the action of the agonist, while an inverse agonist produces an effect opposite to that of the agonist. Receptor antagonists are receptor ligands or drugs that block or dampen a biological response, which is normally induced by the receptor, by binding to and blocking a receptor rather than activating it like an agonist. Antagonistic drugs interfere in the natural operation of receptor proteins. In pharmacology, antagonists can have an affinity for their cognate receptors but induce no efficacy, as binding to the receptor only disrupts the interaction and inhibits the function of an agonist or inverse agonist at receptors. Antagonists produce their effect by binding to the active site or allosteric site of a receptor, or they may interact at unique binding sites not normally involved in the biological regulation of a receptor's activity. Antagonist activity may be reversible or irreversible.

Herein “receptors” are cellular proteins whose activation causes a downstream biological response and/or induces a downstream biological signaling cascade and/or process. Receptors can be activated by either endogenous agonists (such as hormones and neurotransmitters) or exogenous agonists (such as drugs), resulting in a biological response. Binding of an agonist to a receptor occurs as a result of non-covalent interactions at locations called the binding site on the receptor. A receptor may contain one or more binding sites for different ligands. Commonly receptors are large protein molecules that can be membrane-bound, cell surface receptors, or inside the cell as intracellular receptors, e.g., nuclear receptors.

A “pharmacophore” is considered in the field of biochemistry to be an ensemble of steric and electronic features that is necessary to ensure the optimal supramolecular interactions with a specific biological target and to activate (or inhibit) its biological response. In embodiments, a pharmacophore may be considered an abstract definition of molecular features required for the molecular recognition of a ligand by a biological macromolecule, such as a receptor.

In some embodiments, a ligand binds specifically to its target. “Specific binding” is to be understood as via one skilled in the art, whereby the skilled person is clearly aware of various experimental procedures that can be used to test binding and binding specificity. Methods for determining equilibrium association or equilibrium dissociation constants are known in the art. Some cross-reaction or background binding may be inevitable in many protein-protein interactions; this is not to detract from the “specificity” of the binding between a ligand and its target. “Specific binding” describes binding of a ligand to its class B1 G protein-coupled receptor target at greater binding affinity than background binding. The term “directed against” is also applicable when considering the term “specificity” in understanding the interaction between ligand and target.

Herein a “sample” may be any relevant sample for analysis, such as (without limitation) a sample taken from a patient, a cell culture of patient cells or cell lines, an animal, or a cell culture of animal cells or cell lines of a biopsy, a blood sample, a tissue sample, an environmental sample, or a food-derived sample. A sample may be any kind of biological sample. As used herein, the term “sample” is preferably a biological sample that is obtained or isolated from a patient, a subject, an animal, the environment or any other biological source. In some embodiments a sample may refer to a sample of bodily tissue or fluid, such as a liquid biopsy, blood, serum, plasma, cerebrospinal fluid, urine, saliva, sputum, pleural effusions, cells, a cellular extract, a tissue sample, a tissue biopsy, an organ, a stool sample and the like.

Herein the terms “subject” or “patient” may be used interchangeably. Herein as subject, or patient may be an organism, a cell culture of patient cells or cell lines, an animal, or a cell culture of animal cells or cell lines. Herein a subject or patient refers to a species from which a sample is taken, and/or whose biological material makes up the majority of the biological material of a sample. A subject or patient can be selected from the group comprising vertebrae, animals, livestock, mammals, humans, preferably mammal or human.

Herein, “nucleic acid” may preferably refer to DNA (deoxyribonucleic acid), gDNA (genomic deoxyribonucleic acid), RNA (ribonucleic acid), gRNA (genomic ribonucleic acid), mRNA (messenger ribonucleic acid) and cDNA (complementary deoxyribonucleic acid synthesized from RNA template), or any combination thereof. In a preferred embodiment the nucleic acid herein is DNA. Nucleic acid sequences refer herein to a consecutive array of nucleotides, wherein the nucleotides are distinguished by their nucleobases into guanine (G), adenine (A), cytosine (c) and thymine (T) in DNA and uracil (U)-instead of thymine-in RNA. A nucleic acid sequence may herein also refer to the sequence of consecutive letters (comprised of G, A, C and T or U) that represent the actual sequence of consecutive nucleic acids in a strand of DNA or RNA. This nucleic acid sequence may be biochemically and bioinformatically identified and characterized using DNA or RNA sequencing. The sequencing analysis may also involve the comparison of the obtained nucleic acid sequence and one or more reference nucleic acid sequences. As used herein, “nucleic acid” shall mean any nucleic acid molecule, including, without limitation, DNA, RNA and hybrids or modified variants thereof. An “exogenous nucleic acid” or “exogenous genetic element” relates to any nucleic acid introduced into the cell, which is not a component of the cells “original” or “natural” genome. Exogenous nucleic acids may be integrated or non-integrated or relate to stably transfected nucleic acids.

“Amino acids” (AA, aa) are chemical compounds with a nitrogen (N) containing amino group and a carbon (C) and oxygen (O) containing carboxylic acid group. The class of amino acids includes organic compounds that contain at least one amino group (—NH₂ or substituted —NR₂) and one carboxy group (—COOH) as functional groups. Selected α-amino acids are the natural building blocks of proteins, which are linked together to form chains by the carboxy group of one amino acid forming a peptide bond with the amino group of the next. The amino acids thus linked to form a polymer differ in their side chains and together determine the shape with which the polypeptide then unfolds in the aqueous environment to form the native protein. This biosynthesis of proteins takes place in all cells at the ribosomes according to genetic information, which is available in the form of mRNA. The base sequence of the mRNA encodes the amino acid sequence in triplets, with one base triplet each representing a codon that stands for a specific proteinogenic amino acid.

A “peptide” is an organic compound containing peptide bonds between amino acids. Accordingly, a “peptide” or “peptide sequence” refers to the sequence (and/or identity) of consecutively linked amino acids of a peptide. According to their number, oligopeptides with few are distinguished from polypeptides with many amino acids. Long polypeptide chains are also called “proteins”, especially those formed by protein biosynthesis. As used herein, “polypeptide” shall mean both peptides and proteins. In this invention, the polypeptides may be naturally occurring or recombinant (i.e., produced via recombinant DNA technology), and may contain mutations (e.g., point, insertion and deletion mutations) as well as other covalent modifications (e.g., glycosylation and labelling (via biotin, streptavidin, fluorescein, and radioisotopes)), non-proteinogenic amino acids, artificial amino acids and/or other molecular bonds to additional components.

A “non-proteinogenic amino acid” or non-coded amino acid is distinct from the 22 proteinogenic amino acids that are naturally encoded in the genome of organisms for the assembly of proteins.

In principle any organic compound with an amine (—NH₂) and a carboxylic acid (—COOH) functional group is an amino acid. Nonproteinogenic amino acids can be incorporated into proteins with non-ribosomal peptide synthetases, by protein ligation, by peptide synthesis, or by genetic reprogramming. An example of a non-proteinogenic amino acid is 2-aminoisobutyric acid.

Sequence variants of the claimed conjugates, nucleic acids, proteins and/or other biomolecules, for example defined by the claimed % sequence identity, that maintain the said properties of the invention, are also included in the scope of the invention. Such variants, which show alternative sequences, but maintain essentially the same binding properties, such as target specificity, as the specific sequences provided are known as functional analogues, or as functionally analogous.

Sequence identity relates to the percentage of identical nucleotides or amino acids when carrying out a sequence alignment.

Protein sequence modifications, which may occur through substitutions, are also included within the scope of the invention. Substitutions as defined herein are modifications made to the amino acid sequence of the protein, whereby one or more amino acids are replaced with the same number of (different) amino acids, producing a protein which contains a different amino acid sequence than the primary protein, preferably without significantly altering the function of the protein. Like additions, substitutions may be natural or artificial. It is well known in the art that amino acid substitutions may be made without significantly altering the protein's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the protein. Frequently, many amino acids may be substituted by conservative amino acids without deleteriously affecting the protein's function.

In general, the non-polar amino acids Gly, Ala, Val, Ile and Leu; the non-polar aromatic amino acids Phe, Trp and Tyr; the neutral polar amino acids Ser, Thr, Cys, Gln, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups. Substitution variants have at least one amino acid residue in the antibody molecule removed and a different residue inserted in its place.

| | Preferred | |
|---|---|---|
| Original | conservative | |
| residue | substitutions | Examples of exemplary substitutions |
| Ala (A) | Val | Val; Leu; Ile |
| Asg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn, Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; Norleucine |

Conservative amino acid substitutions are not limited to naturally occurring amino acids, but also include synthetic amino acids. Commonly used synthetic amino acids are omega amino acids of various chain lengths and cyclohexyl alanine which are neutral non-polar analogs; citrulline and methionine sulfoxide which are neutral non-polar analogs, phenylglycine which is an aromatic neutral analog; cysteic acid which is a negatively charged analog and ornithine which is a positively charged amino acid analog. Like the naturally occurring amino acids, this list is not exhaustive, but merely exemplary of the substitutions that are well known in the art.

Variants of proteins: “Variants” of proteins or peptides, as defined in the context of the present invention, may be generated that have an amino acid sequence that differs from the original sequence in one or more mutation(s), such as one or more substituted, inserted and/or deleted amino acid(s). In embodiments, the sequence variation described here with respect to conservative substitutions and/or percentage identity, may apply to any one or more embodiments, described throughout the application as a whole.

Preferably, these fragments and/or variants have the same biological function or specific activity compared to the native full-length protein, e.g., its specific antigenic property. “Variants” of proteins or peptides, as defined in the context of the present invention, may comprise one or more conservative amino acid substitution(s) compared to their native, i.e., non-mutated, physiological sequence. These amino acid sequences, as well as their coding nucleotide sequences, more particularly fall within the term “variants” as defined herein. Substitutions in which amino acids originating from the same class are exchanged for each other are called conservative substitutions. These are in particular amino acids with aliphatic side chains, positively or negatively charged side chains, aromatic groups in the side chains or amino acids whose side chains can form hydrogen bonds, e.g., side chains that have a hydroxyl function. This means that, for example, an amino acid with a polar side chain is replaced by another amino acid with a side chain that is also polar, or, for example, an amino acid characterised by a hydrophobic side chain is replaced by another amino acid with a side chain that is also hydrophobic (e.g., serine (threonine) by threonine (serine) or leucine (isoleucine) by isoleucine (leucine)).

Insertions and substitutions are possible especially at such sequence positions that do not cause a change in the three-dimensional structure or do not affect the binding region. Modifications to a three-dimensional structure by insertion(s) or deletion(s) can be easily determined, e.g., using circular dichroism spectra (CD spectra) (Urry, 1985, Absorption, Circular Dichroism and ORD of Polypeptides, in: Modern Physical Methods in Biochemistry, Neuberger et al., (eds.), Elsevier, Amsterdam).

Furthermore, variants of proteins or peptides as defined herein that may be encoded by a nucleic acid molecule may also comprise such sequences, wherein nucleotides of the encoding nucleic acid sequence are exchanged according to the degeneracy of the genetic code without any change in the respective amino acid sequence of the protein or peptide, i.e. the amino acid sequence or at least a part thereof may not differ from the original sequence in one or more mutation(s) as defined above.

Fragments of proteins: “Fragments” of proteins or peptides in the context of the present invention may typically comprise a sequence of a protein or peptide as defined herein, that is, with respect to its amino acid sequence (or its encoded nucleic acid) molecule), N-terminally and/or C-terminally truncated compared to the amino acid sequence of the original (native) protein (or its encoded nucleic acid molecule).

In this context, a fragment of a protein may typically comprise an amino acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98% or 99%, preferably of at least 70%, more preferably of at least 80%, even more preferably of at least 85%, even more preferably of at least 90% and most preferably of at least 95% or even 97%, having an amino acid sequence of the respective naturally occurring full-length protein.

It is understood that substituents and substitution patterns of the compounds described herein can be selected by one of ordinary skill in the art to provide compounds that are chemically stable and that can be readily synthesized by techniques known in the art and further by the methods set forth in this disclosure.

Variation in length of the amino acid sequences and encoding nucleic acids as described herein is also encompassed by the present invention. A skilled person is capable of providing natural or artificial amino acid sequence variants that are longer or shorter than the specific sequences of SEQ ID NO 1 to 20, which will still exhibit sufficient similarity to the natural proteins in order to provide the desired binding capabilities described herein. For example, shorter variants of the amino acid sequences of the present peptide conjugates, e.g., of SEQ ID NO 1 to 4 or of SEQ ID NO 1 to 20 comprising 1, 2, 3, 4, 5, 10, 15, 20, 25, 30, 35, 40, 45 or 50 amino acids less than the full-length form may also exhibit effective binding capabilities, as described herein. For example, longer variants of the of the present peptide conjugates, e.g., of SEQ ID NO 1 to 4 or of SEQ ID NO 1 to 20, comprising 1, 2, 3, 4, 5, 10, 15, 20, 25, 30, 35, 40, 45 or 50 amino acids of a class B1 G protein-coupled receptor-protein ligand more than the natural length form may also exhibit effective binding capabilities, as described herein.

In some embodiments of the invention the peptide, preferably according to sequences disclosed herein, may comprise a 0 to 10 amino acid addition or deletion at the N and/or C terminus of a sequence.

As used herein the term “a 0 to 10 amino acid addition or deletion at the N and/or C terminus of a sequence” means that the polypeptide may have a) 0, 1, 2, 3, 4, 5, 6, 7, 8, 9 or 10 additional amino acids at its N terminus and 0, 1, 2, 3, 4, 5, 6, 7, 8, 9 or 10 amino acids deleted at its C terminus or b) 0, 1, 2, 3, 4, 5, 6, 7, 8, 9 or 10 additional amino acids at its C terminus and 0, 1, 2, 3, 4, 5, 6, 7, 8, 9 or 10 nucleotides deleted at its N terminus, c) 0, 1, 2, 3, 4, 5, 6, 7, 8, 9 or 10 additional amino acids at its N terminus and 0, 1, 2, 3, 4, 5, 6, 7, 8, 9 or 10 additional amino acids at its N terminus or d) 0, 1, 2, 3, 4, 5, 6, 7, 8, 9 or 10 amino acids deleted at its N terminus and 0, 1, 2, 3, 4, 5, 6, 7, 8, 9 or 10 amino acids deleted at its C terminus.

As used herein, the terms “protein ligand comprising an amino acid sequence with least 70% identity to an endogenous human protein ligand of at least one class B1 G protein-coupled receptor” and “peptide comprising an amino acid sequence of an agonist, antagonist and/or a co-agonist of at least one class B1 G protein-coupled receptor, or a sequence with least 70% identity to said agonist, antagonist and/or a co-agonist” includes a protein or peptide that has at least about 50% amino acid identity with one or more endogenous human protein ligand(s) of at least one class B1 G protein-coupled receptor, wherein the ligand is preferably an agonist, antagonist and/or a co-agonist of at least one class B1 G protein-coupled receptor, e.g., GLP-1, GIP, glucagon or a precursor protein thereof, or dual and triple agonists of class B1 G protein-coupled receptors comprising sequences of GLP-1, GIP or glucagon, or combinations thereof (e.g., tirzepatide, LY3437943). As a non-limiting example, an isoform of an endogenous human protein ligand of at least one class B1 G protein-coupled receptor according to the invention can have at least about 55%, 60%, 65%, 70%, 75%, 80%, 85%, 90%, 91%, 92%, 93%, 94%, 95%, 96%, 97%, 98%, or 99% amino acid sequence identity with one or more of e.g., GLP-1, GIP, glucagon or a precursor protein thereof, or a dual and triple agonists comprising sequences of GLP-1, GIP or glucagon, or combinations thereof (e.g., tirzepatide, LY3437943). Nucleic acid variants to, e.g., GLP-1, GIP, glucagon or a precursor protein thereof, or dual and triple agonists comprising sequences of GLP-1, GIP or glucagon, or combinations thereof (e.g., tirzepatide, LY3437943) and SEQ ID NO 1-4 or SEQ ID NO 1 to 20 are also encompassed herein that encode an amino acid sequence of GLP-1, GIP, glucagon or a precursor protein thereof, or dual and triple agonists comprising sequences of GLP-1, GIP or glucagon or combinations thereof e.g. tirzepatide, LY3437943) or SEQ ID NO 1-4 or SEQ ID NO 1 to 20, or a sequence with substantially the same amino acid sequence. The complementary nucleic acid sequence is also encompassed, as is a degenerate sequence modified to use the degenerate nature of the genetic code, as is known to a skilled person.

The amino acid sequences may also comprise 0 to 100, 2 to 50, 5 to 20, or for example 8 to 15, or any value from 0 to 50, amino acid additions or deletions at either the N- and/or C-terminus of the proteins. The termini may also be modified with additional linker sequences, or removal of sequences, as long as the receptor binding-properties of the peptide conjugate are essentially maintained.

Various ways of preparing functionally analogous peptides have been disclosed in the prior art and a known to a skilled person. Peptides designed starting from the peptides of the invention using such methods are included in the teaching according to the invention. Hence, all peptides, peptide fragments or structures comprising peptides generated using the methods mentioned above, starting from the peptides or peptide conjugates according to the invention, are considered peptides or peptide conjugates according to the invention, provided they accomplish the object of the invention and, in particular, interact with/bind at least one class B1 G protein-coupled receptor as protein ligand, wherein the ligand is preferably an agonist, antagonist and/or a co-agonist of at least one class B1 G protein-coupled receptor.

The instant disclosure also includes kits and packages containing the herein described peptide conjugates and/or nucleic acids encoding the same, and/or means for preparing and/or using the same.

As used herein, a “label” is any compound, substance, molecule, atom, chemical modification or any other entity or structure, that makes possible the identification or determination of the conjugate, for example and without limitation, in any analytical and/or biological system. In embodiments, the label may be considered a “tag” or “marker” that facilitates detection and/or purification. A variety of methods are available to generate labeled peptides. In embodiments, a “label” preferably comprises a fluorescent or luminescent label, a nucleic acid label, a peptide label, an antibody or antigen-binding fragment thereof, a biotin label, a chromogenic label, an MRI label, a metal label, dyes, radionuclides, or iodine-125 (125I). Fluorescence is a form of luminescence that occurs when matter emits light of a certain wavelength after absorbing electromagnetic radiation.

A “fluorophore” (or fluorochrome, similar to a chromophore) is a fluorescent chemical compound that can reemit light when excited by light. Fluorophores for use as fluorescent or luminescent labels include, without being limited thereto, rhodamine and derivatives, lissamine fluorescein, 5-bromomethylfluorescein and derivatives, DAPI, Hoechst 33258, R-phycocyanin, B-phycoerythrin, R-phycoerythrin, Lucifer Yellow, IAEDANS, 7-Me2N-coumarin-4-acetate, 7-OH-4-CH3-coumarin-3-acetate, monobromobiman, Pyrene trisulfonates such as Cascade Blue and monobromotrimethyl ammoniobiman, Texas Red, Rhodamine Green, Oregon Green 30 488, Oregon Green 514, 7-NH₂-4CH3-25-coumarin-3-acetate (AMCA), FAM, TET, CAL Fluor Gold 540, JOE, VIC, Quasar 570, CAL Fluor Orange 560, Cy3, NED, Oyster 556, TMR, CAL Fluor Red 590, HEX, ROX, LC Red 610, CAL Fluor Red 610, Texas Red, LC Red 610, CAL Fluor Red 610, LC Red 640, CAL Fluor Red 635, Cy5, LC Red 670, Quasar 670, Oyster 645, LC Red 705, Cy5.5, BODIPY FL, Cal Gold, BODIPY R6Gj, Yakima Yellow, Cal Orange, BODIPY TMR-X, JOE, HEX, Quasar-570/Cy3, TAMRA, Rhodamine Red-X, Redmond Red, BODIPY 581/591, Cy3.5, Cal Red/Texas Red, BODIPY 630/665-X, BODIPY TR-X, Quasar-670/Cy5, Pulsar-650, Cy5, Cy5.5, Dy590, Dy490, Dy636, Dy682, Atto-488, Atto532, Atto-Rho-6G, Atto-Rho101, Atto-647N, Atto-680, BMN-488, BMN-505, BMN-536, BMN-562. Indirect labels, such as chromogenic labels, include various enzymes well-known in the art, such as horseradish peroxidase (HRP), alkaline phosphatase (AP), β-galactosidase, urease, and the like.

Fluorescence imaging comprises various techniques for the visualization of fluorescent proteins or fluorescence dyes as markers of biological structures and functions. Fluorescence imaging facilitates the detection, monitoring and research of, for example, molecular functions and processes, such as the localization or presence of gene/protein expression, protein-protein interactions and molecular signaling pathways in the cellular and tissue context. Fluorescence imaging comprises techniques such as microscopy, Fluorescence Activated Cell Sorting (FACS), imaging probes, and spectroscopy.

During fluorescence microscopy the specimen is illuminated with a specific wavelength(s) of light that can be absorbed by the fluorophore(s) such that the fluorophore(s) emit light of longer wavelengths. Preferably, in the microscope a spectral emission filter separates the excitation light from the fluorescence emitted from the specimen, which is commonly much weaker.

In embodiments, a “label” may be or may comprise an antibody or an antigen binding fragment thereof. As used herein, an “antibody” generally refers to a protein consisting of one or more polypeptides substantially encoded by immunoglobulin genes or fragments of immunoglobulin genes. Where the term “antibody” is used, the term “antibody fragment” may also be considered to be referred to. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. The basic immunoglobulin (antibody) structural unit is known to comprise a tetramer or dimer. Each tetramer is composed of two identical pairs of polypeptide chains, each pair having one “light” (L) (about 25 kD) and one “heavy” (H) chain (about 50-70 kD). The N-terminus of each chain defines a variable region of about 100 to 110 or more amino acids, primarily responsible for antigen recognition. The terms “variable light chain” and “variable heavy chain” refer to these variable regions of the light and heavy chains respectively. Optionally, the antibody or the immunological portion of the antibody, can be chemically conjugated to, or expressed as, a fusion protein with other proteins.

Antibodies or antibody fragments of the invention therefore include, but are not limited to polyclonal, monoclonal, bispecific, human, humanized or chimeric antibodies, single chain fragments (scFv), single variable fragments (ssFv), single domain antibodies (such as VHH fragments from nanobodies), Fab fragments, F(ab′)₂ fragments, fragments produced by a Fab expression library, anti-idiotypic antibodies and epitope-binding fragments or combinations thereof of any of the above, preferably comprising the corresponding CDRs, or VH and VL regions as described herein. Also mini-antibodies and multivalent antibodies such as diabodies, triabodies, tetravalent antibodies and peptabodies can be used in the invention. The immunoglobulin molecules of the invention can be of any class (i.e. IgG, IgE, IgM, IgD and IgA) or subclass of immunoglobulin molecules

The detection of a label that is or comprises an antibody or an antigen-binding fragment thereof, can in embodiments be achieved through the detection of said antibody with a secondary antibody. Either the antibody or antigen-binding fragment thereof comprised within the label and/or the secondary antibody can in embodiments be coupled to a dye or an enzyme, such as horseradish peroxidase (HRP), alkaline phosphatase (AP), β-galactosidase, urease, and the like. The skilled person is aware of methods and means to detect an antibody label and/or link an antibody to a dye or enzyme.

### FIGURES

The invention is further described by the following figures. These are not intended to limit the scope of the invention but represent preferred embodiments of aspects of the invention provided for greater illustration of the invention described herein.

### DETAILED DESCRIPTION OF THE FIGURES

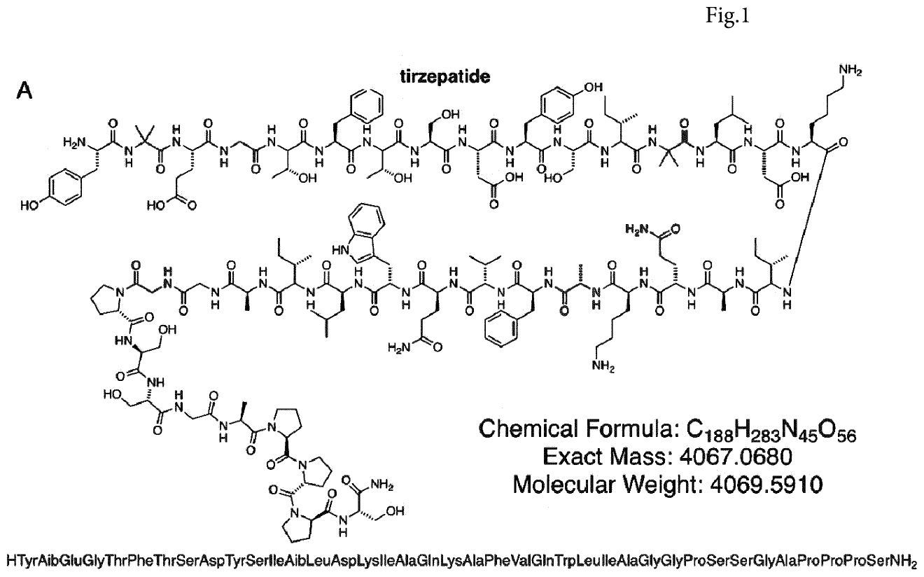

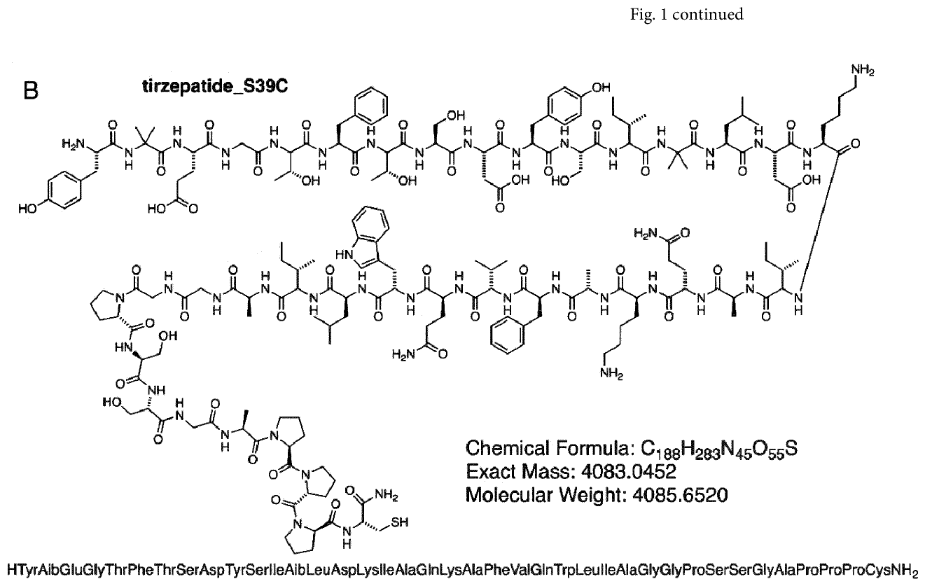

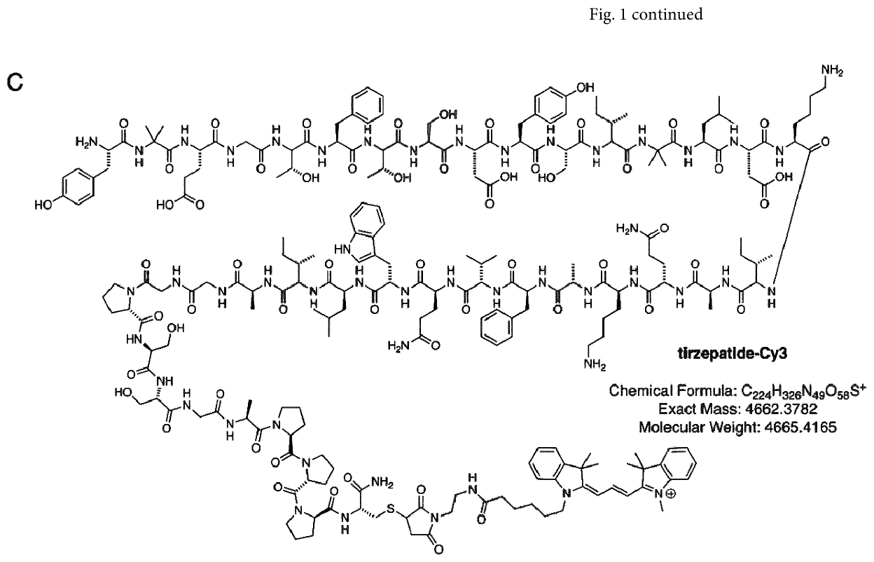

FIG. 1: (A) Structure of tirzepatide (SEQ ID NO 5), (B) Structure of tirzepatide modified with a C-terminal Cysteine (SEQ ID NO 6), (C) Structure of tirzepatide C-terminally linked to Cy3 (SEQ ID NO 3), (D) Structure of an embodiment of a peptide conjugate of tirzepatide C-terminally linked to Cy5 (SEQ ID NO 4). This Figure illustrates one embodiment of a conjugation strategy according to the invention for the preparation of one embodiment of a peptide conjugate according to the invention.

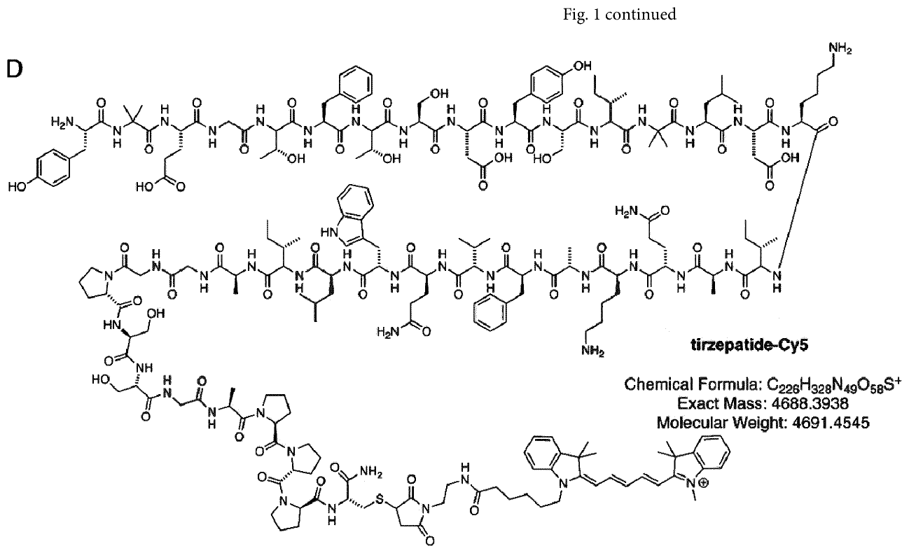

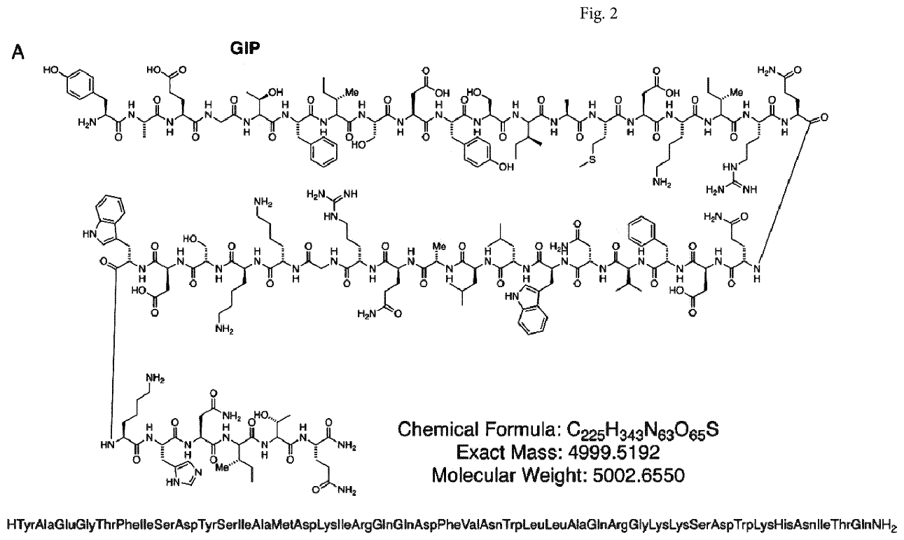

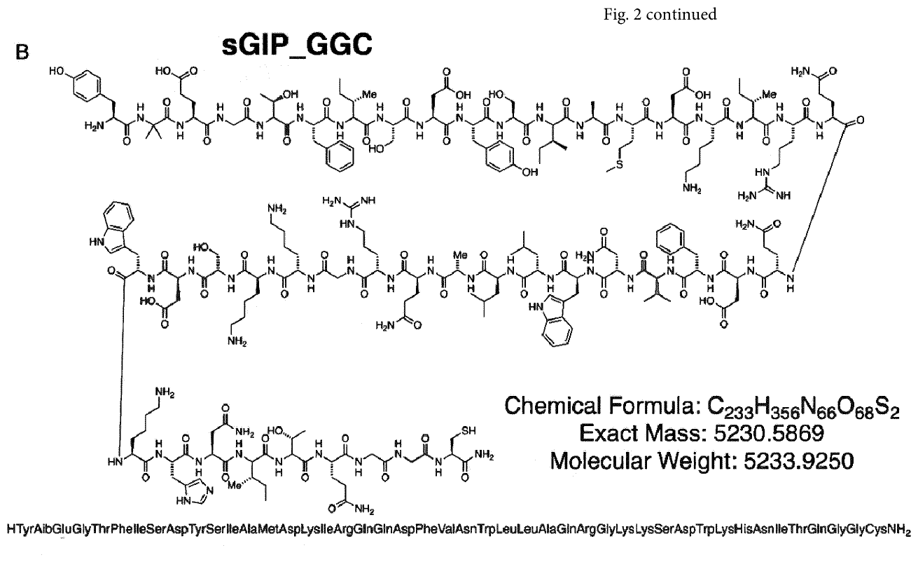

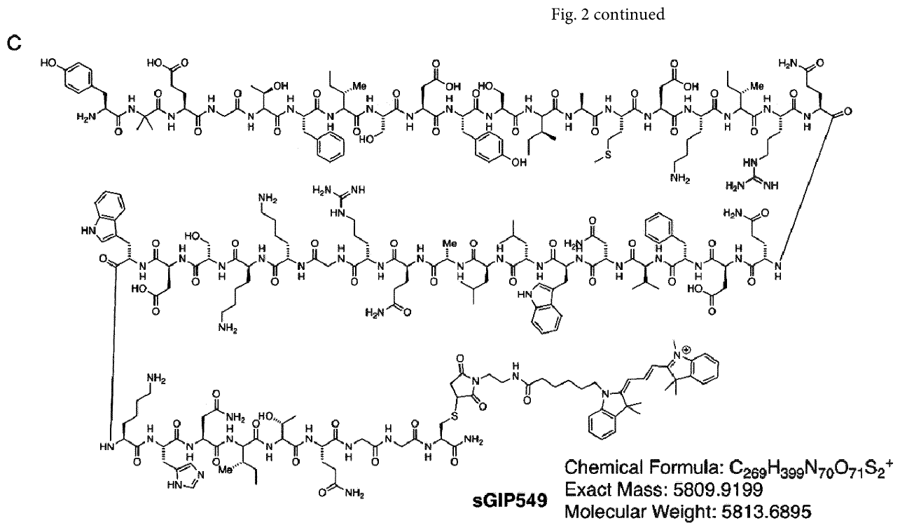

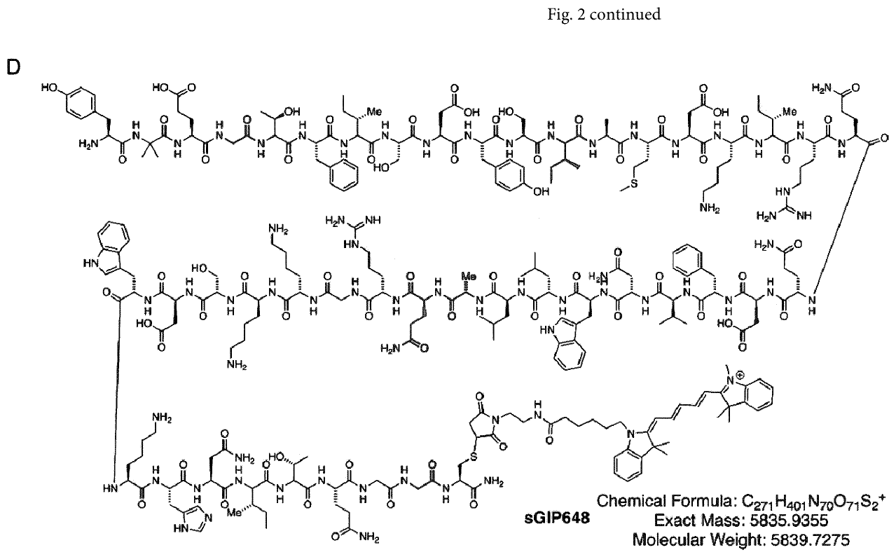

FIG. 2: (A) Structure of GIP (SEQ ID NO 11), (B) Structure of GIP C-terminally modified with two Glycines and a terminal Cysteine (SEQ ID NO 8), (C) Structure of GIP C-terminally linked to Cy3 (SEQ ID NO 1), (D) Structure of an embodiment of a peptide conjugate of GIP C-terminally linked to Cy5 (SEQ ID NO 2). This Figure illustrates one embodiment of a conjugation strategy according to the invention for the preparation of one embodiment of a peptide conjugate according to the invention.

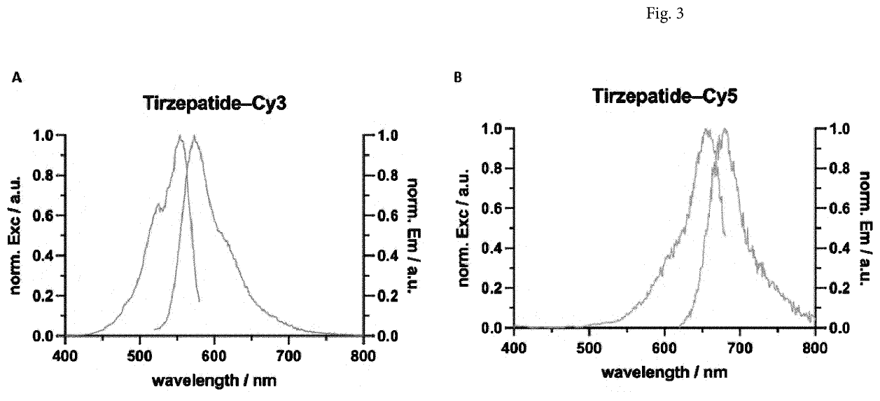

FIG. 3: Characterization of tirzepatide peptide conjugates. (A) Tirzepatide-Cy3(Mal) was prepared according to general procedure described herein. HRMS (QT of): calc. for C₂₂₄H₃₂₉N₄₉O₅₈S [M+3H]⁴⁺:1166.8517, found: 1166.8503. t_{R} (QT of; MeCN/H₂O/formic acid=5/95/0.1 to 95/5/0.1 over 10 min)=4.69 min. (B) Tirzepatide-Cy5(Mal) was prepared according to general procedure described herein above. HRMS (QT of): calc. for C₂₂₆H₃₃₁N₄₉O₅₈S [M+3H]⁴⁺: 1173.3556, found: 1173.5955. t_{R} (QT of; MeCN/H₂O/formic acid=5/95/0.1 to 95/5/0.1 over 10 min)=4.74 min.

FIG. 4: Characterization of sGIP peptide conjugates. (A) sGIP-Cy3 was prepared according to general procedure described herein above. LRMS: calc. for C₂₆₉H₄₀₄N₇₀O₇₁S₂ [M+5H]⁶⁺: 969.5, found: 969.7. (B) sGIP-Cy5 was prepared according to general procedure described herein. LRMS: calc. for C₂₇₁H₄₀₇N₇₀O₇₁S₂ [M+6H]⁷⁺: 835.1, found: 835.5.

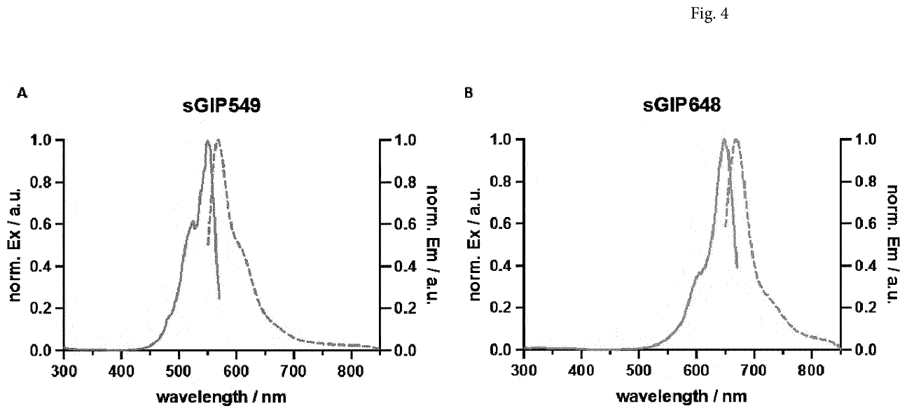

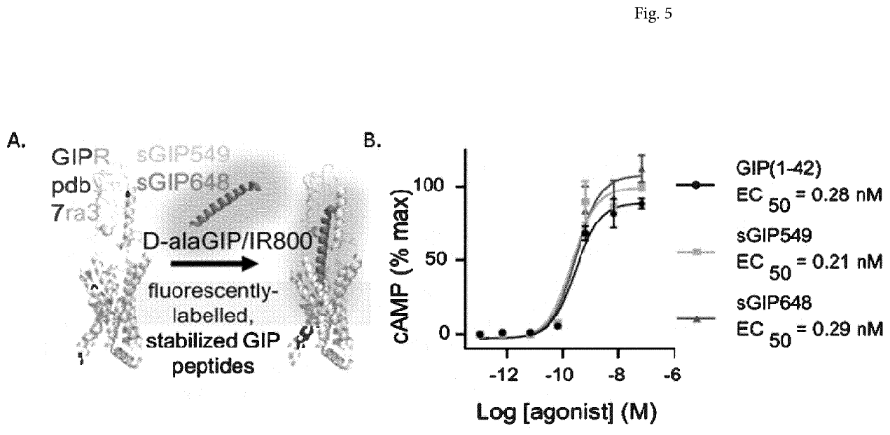

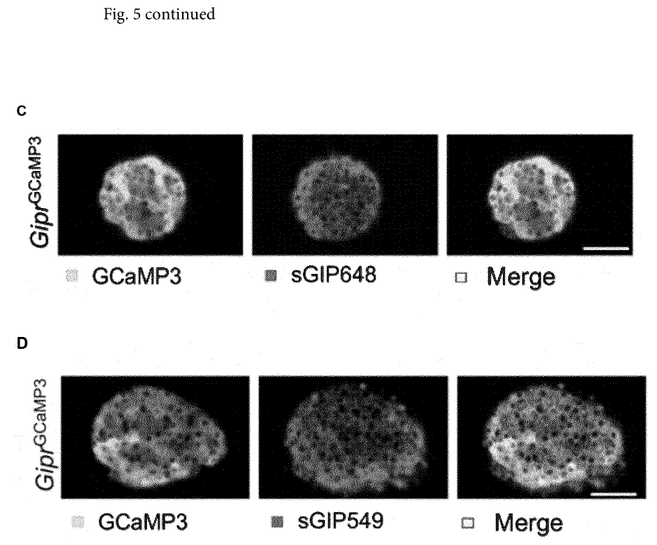

FIG. 5: Stabilized, fluorescently-labelled GIP peptides are specific and effective GIPR agonists. (A) Schematic showing nature and binding of the stabilized red (sGIP549) and far red (sGIP648) GIPR probes (GIPR pdb: 7ra3). (B) sGIP549, sGIP648 and native GIP(1-42) cAMP signaling responses in T-REx-SNAP-GIPR cells, n=3. (C)-(D) sGIP648 (C) and sGIP549 (D) label GIPR-GCAMP3 reporter islets, showing co-localization with GCAMP3+ cells (n=11-12 islets, 2 animals).

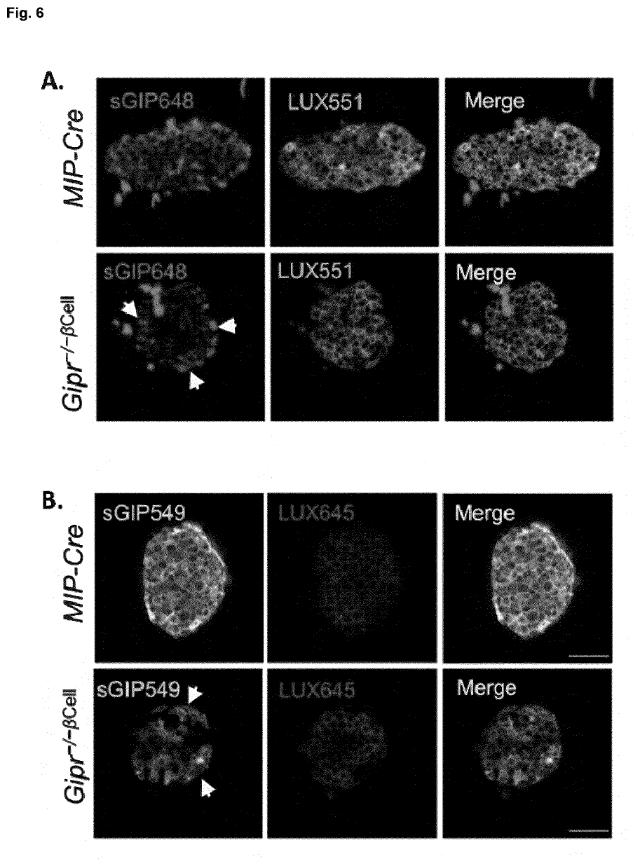

FIG. 6 Stabilized, fluorescently-labelled GIP peptides allow new insight into GIPR expression in the pancreatic islet. (A) sGIP648 labels beta cells, identified using LUX551, in Mip-Cre but not Gipr^{-/-}βcell islets. Arrows show GIPR labelling only in LUX551-negative cells, presumed to be alpha cells (n=63 islets, 10 animals). (B) sGIP549 labels beta cells, identified using LUX645, in Mip-Cre but not Gipr^{-/-}βcell islets. Arrows show GIPR labelling only in LUX645-negative cells, presumed to be alpha cells (n=56 islets, 10 animals).

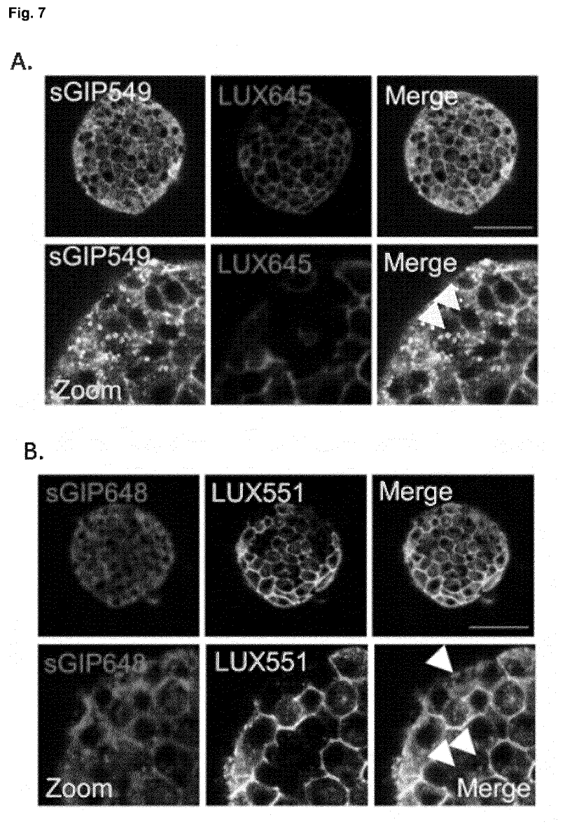

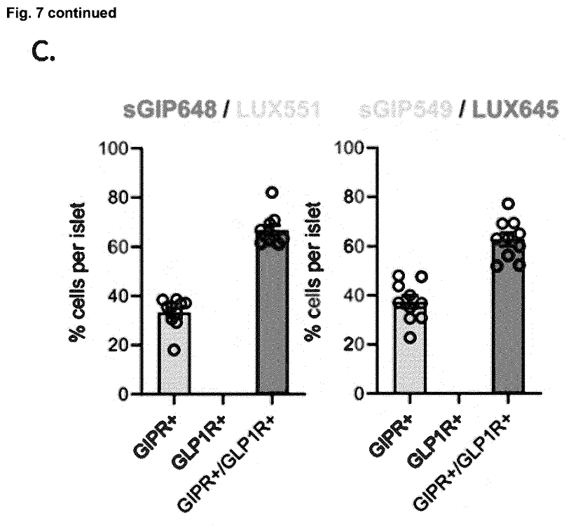

FIG. 7: (A)-(C) sGIP549 (A) and sGIP648 (B) label GIPR+ cells (presumed alpha cells, which are more abundant at the islet surface) and GIPR+ GLP1R+ cells (presumed to be beta cells, since alpha cells do not express Glp1r). As expected, all GLP1R+ cells are GIPR+(C). Arrows show cells that express only GIPR. Scale bar=53 μm. Bar graphs shown mean±SEM.

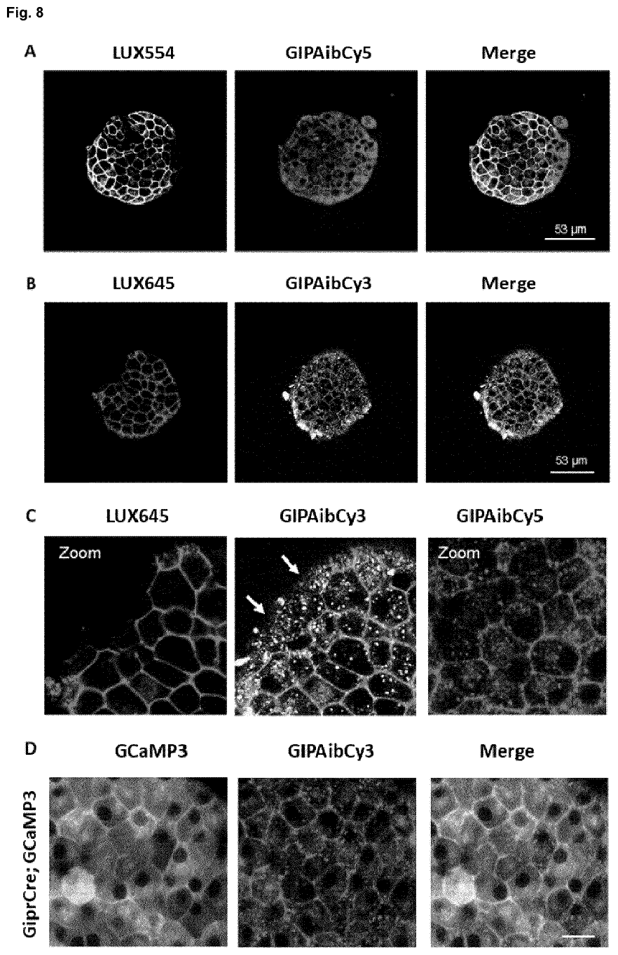

FIG. 8: Confocal imaging of live islets of Langerhans using the peptide conjugates according to the present invention. (A) First row: Confocal imaging of live islets of Langerhans using LUX554 (Ast et al., Nat. Commun. 2020) and JB2229 (GIPAibCy5). (B) Second row: Confocal imaging of live islets of Langerhans using LUX645 (Ast et al., Nat. Commun. 2020) and JB2228 (GIPAibCy3). (C) Third row: zoom-in of second row shows internalized structures, as expected for peptidic agonists. (D) Fourth row: Confocal imaging of live islets of Langerhans expressing GCaMP3 under the GIPR promoter and stained with JB2228 (GIPAibCy3).

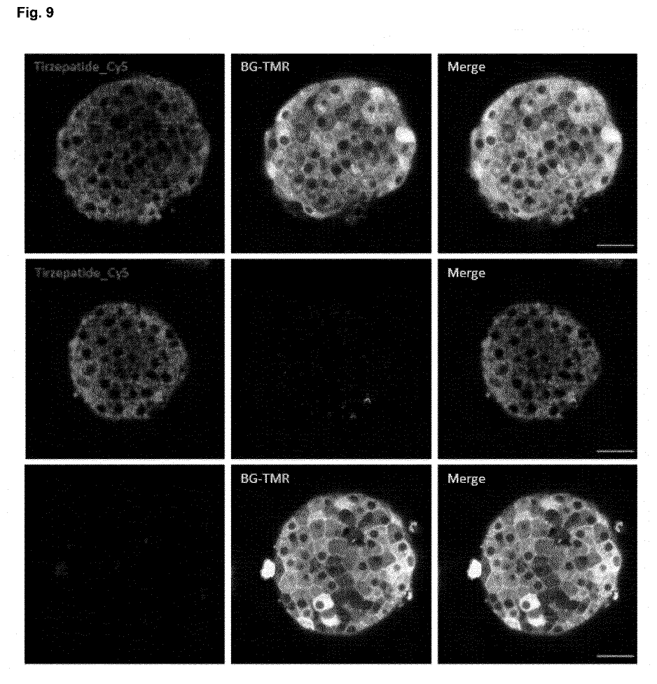

FIG. 9: Confocal microscopy of GLP1R^{SNAP/SNAP} islets stained with one embodiment of the present peptide conjugates, namely a Tirzepatide_Cy5 (TP_Cy5) conjugate. SNAP-GLP1R islets were stained four days after isolation using 500 nM TP_Cy5 (left panel) and 500 nM BG-TMR (middle panel). The right panel shows the merged images. The staining was performed for 1 h, at 37° C., 5% CO₂ in complete media, cells were washed 3× in complete media and imaged in complete media in 96-well glass-bottom plate with 63× magnification, the depicted scale bar measures 25 μm.

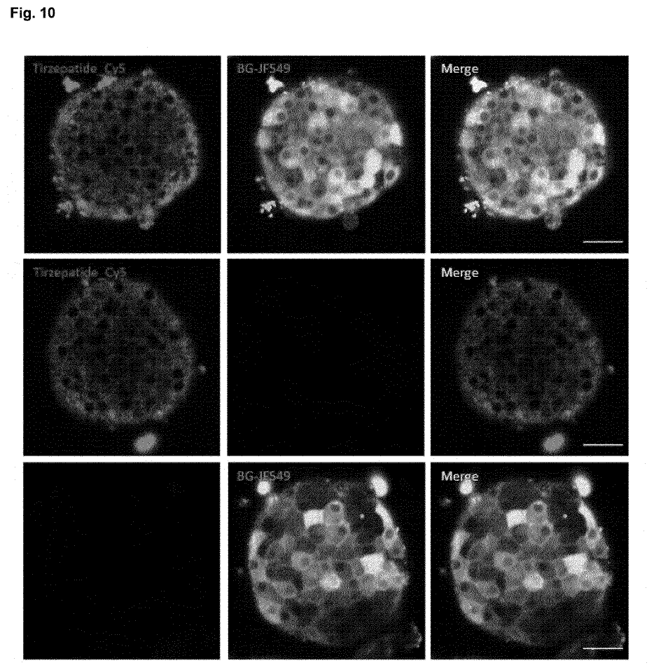

FIG. 10: Confocal microscopy of GLP1R^{SNAP/SNAP} islets stained with one embodiment of the present peptide conjugates, namely a Tirzepatide_Cy5 (TP_Cy5) conjugate. SNAP-GLP1R islets were stained two days after isolation using 500 nM TP_Cy5 (left panel) and 500 nM BG-JF549 (middle panel). The right panel shows the merged images. The staining was performed for 1 h, at 37° C., 5% CO₂ in complete media, cells were washed 3× in complete media and imaged in complete media in 96-well glass-bottom plate with 40× magnification, the depicted scale bar measures 25 μm.

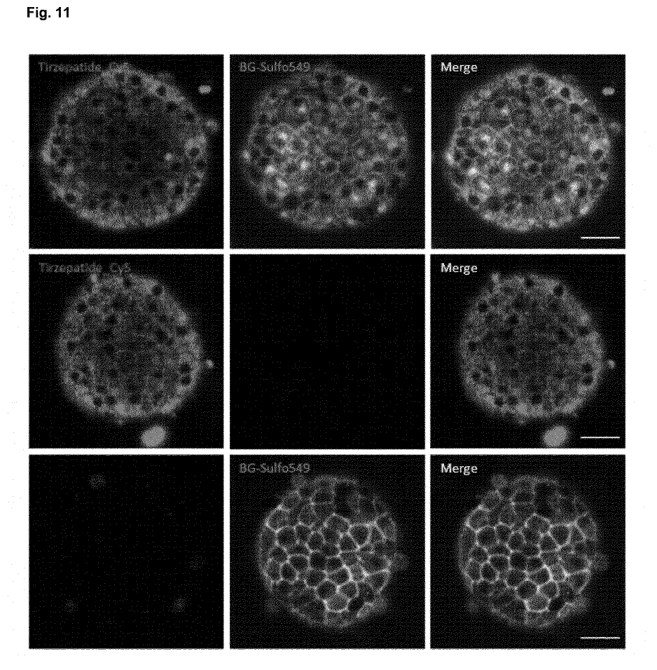

FIG. 11: Confocal microscopy of GLP1R^{SNAP/SNAP} islets stained with one embodiment of the present conjugates, namely a Tirzepatide_Cy5 (TP_Cy5) conjugate. SNAP-GLP1R islets were stained two days after isolation using 500 nM TP_Cy5 (left panel) and 500 nM BG-Sulfo549 (middle panel). The right panel shows the merged images. The staining was performed for 1 h, at 37° C., 5% CO₂ in complete media, cells were washed 3× in complete media and imaged in complete media in 96-well glass-bottom plate with 40× magnification, the depicted scale bar measures 25 μm.

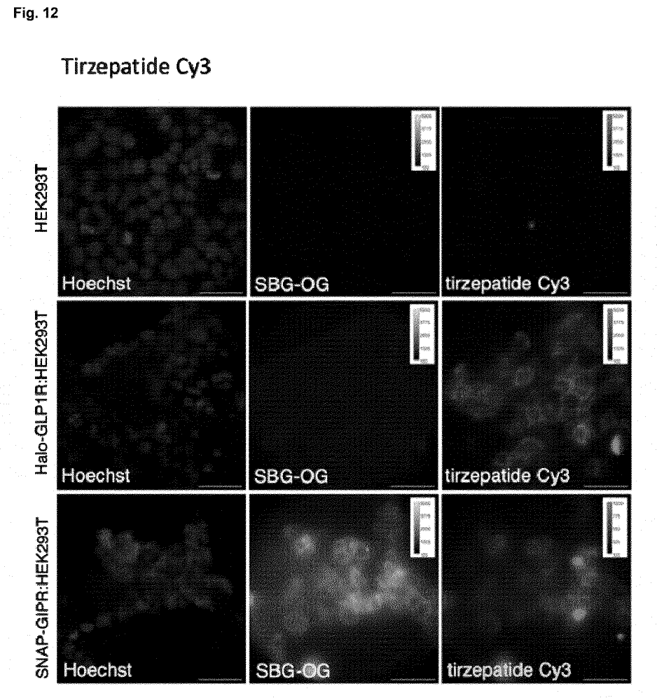

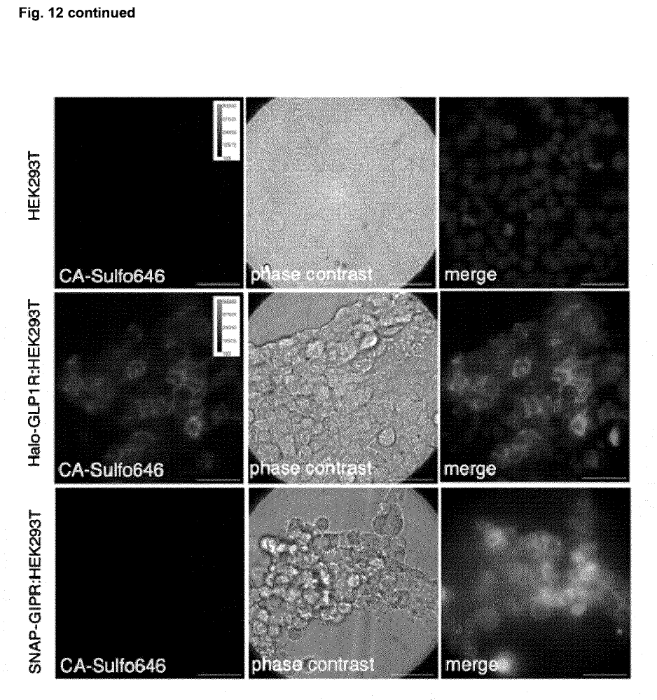

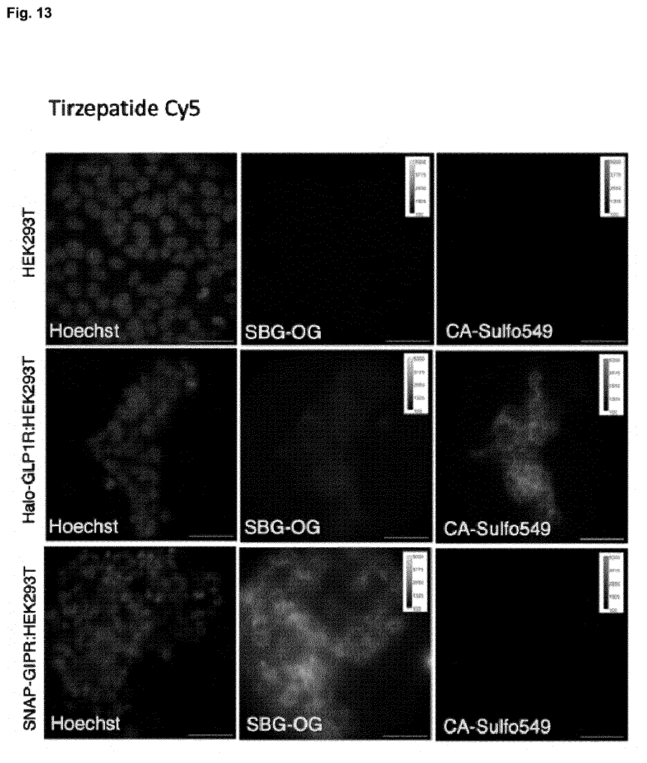

FIG. 12: Epifluorescent microscopy of HEK293T cells transfected with Halo-GLP1R or SNAP-GLP1R stained with embodiments of the peptide conjugates according to the present invention, namely Tirzepatide_Cy3 as antagonists of GLP1R/GIPR. HEK cells were seeded at 75,000 cells/well on PLL-coated μ-well ibidi slide. Transfection was carried with JetPrime 400 ng DNA, staining was performed on the following day in media, using 5 μM Hoechst3342 (far left panel), 500 nM tirzepatide-Cy3 (third panel from left), 1 μM CA-Sulfo646 (third panel from right) or SBG-OG (second panel from left) for 10 min at 37° C., cells were washed once, the image was acquired in fluorobrite at EpiTirf 60× oil.

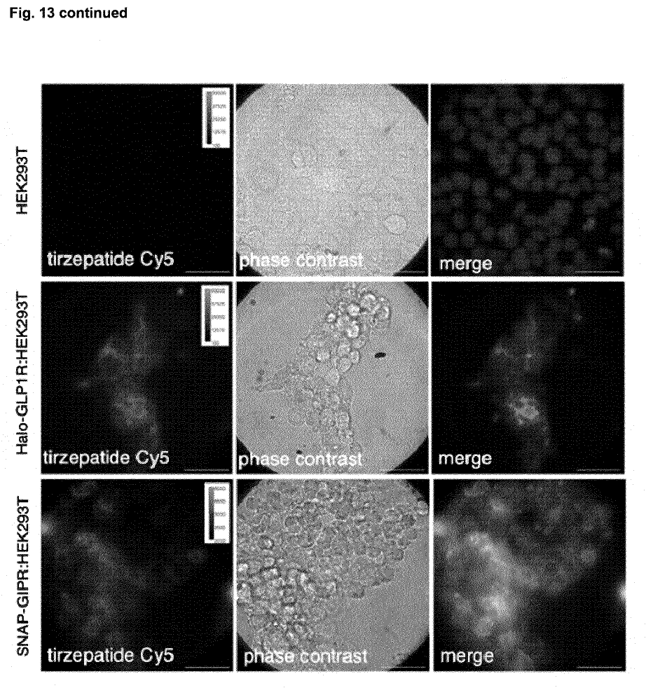

FIG. 13: Epifluorescent microscopy of HEK293T cells transfected with Halo-GLP1R or SNAP-GLP1R stained with embodiments of the peptide conjugates according to the present invention, namely Tirzepatide_Cy5 as antagonists of GLP1R/GIPR. HEK cells were seeded at 75,000 cells/well on PLL-coated μ-well ibidi slide. Transfection was carried with JetPrime 400 ng, staining was performed on the following day in media, using 5 μM Hoechst3342 (far left panel), 500 nM tirzepatide-Cy5 (third panel from right), 1 μM CA-Sulfo549 (third panel from left) or 1 μM SBG-OG (second panel from left) for 10 min at 37° C., cells were washed once, the image was acquired in fluorobrite at EpiTirf 60× oil.

### EXAMPLES

The invention is further described by the following examples. These are not intended to limit the scope of the invention but represent preferred embodiments of aspects of the invention provided for greater illustration of the invention described herein.

In the following examples the inventors demonstrate labelling of endogenous GIPR/GLP1R using various agonist probes conjugated to Cy3 and Cy5 fluorophores. The inventors demonstrate the specificity of their probes in pancreatic islets and cell lines heterologously expressing GIPR/GLP1R.

### General Methods

### General Procedure (“Procedure E”) for Synthesis of Cy5(Mal) and Cy3(Mal) Containing Peptide Labels

The respective peptide was generated using solid phase peptide synthesis. To a solution of the peptide (1.0 equiv.) in PBS (50 μL) was added Cy5- or Cy3-Mal (1.1 equiv.) dissolved in MeCN (50 μL). The solution was allowed to incubate at room temperature (RT) for 8 hours (allowing click chemistry reaction to take place) before being subjected to semipreparative RP-HPLC purification (MeCN:H2O+0.1% TFA=30:70 to 90:10 over 45 minutes).

The purified fractions were combined and lyophilized to yield the Cy5(Mal) or Cy3(Mal) labelled peptide as light blue (Cy5) or light pink (Cy3) TFA salt. The concentration was determined via the absorption of the fluorophore (Cy5 at 647 nm; ε=250,000 mol L-1 cm-1 in DMSO, Cy3 at 546 nm; ε=150,000 mol L-1 cm-1 in DMSO).

### Characterization of Tirzepatide Agonists

Tirzepatide-Cy5(Mal) was prepared according to general procedure described herein above. HRMS (QT of): calc. for C₂₂₆H₃₃₁N₄₉O₅₈S [M+3H]⁴⁺: 1173.3556, found: 1173.5955. t_{R} (QT of; MeCN/H₂O/formic acid=5/95/0.1 to 95/5/0.1 over 10 min)=4.74 min.

Tirzepatide-Cy3(Mal) was prepared according to general procedure described herein above. HRMS (QT of): calc. for C₂₂₄H₃₂₉N₄₉O₅₈S [M+3H]⁴⁺: 1166.8517, found: 1166.8503. t_{R} (QT of; MeCN/H₂O/formic acid=5/95/0.1 to 95/5/0.1 over 10 min)=4.69 min.

Results are shown in FIG. 3.

Characterization of sGIP Agonists

sGIP-Cy3 was prepared according to general procedure described herein above. LRMS: calc. for C₂₆₉H₄₀₄N₇₀O₇₁S₂ [M+5H]⁶⁺: 969.5, found: 969.7.

sGIP-Cy5 was prepared according to general procedure described herein above. LRMS: calc. for C₂₇₁H₄₀₇N₇₀O₇₁S₂[M+6H]⁷⁺:835.1, found: 835.5.

Results are shown in FIGS. 1-4.

### Example 1

Wild-type and GLP1R^{SNAP/SNAP} islets were labelled with sGIP549 (GIPR probe), sGIP648 (GIPR probe), LUXendin645 (GLP1R probe), LUXendin551/554 (GLP1R probe) and Tirzepatide-cy5 (GLP1R/GIPR probe). Live imaging was performed with Zeiss LSM780/LSM880 meta-confocal microscopes equipped with sensitive GaAsP spectral detectors and 40× and 63×/1.2 W Korr FCS M27 objectives. Representative images had linear adjustments applied to brightness and contrast and to enable cross-comparison, intensity values were maintained between samples.

The data clearly show that sGIP549 and sGIP648 label endogenous GIPR, staining areas where LUXendin staining is absent and GLP1R is thus not expressed (i.e., alpha cells that express GIPR and not GLP1R). Tirzepatide-cy5 also labelled the islet, showing overlap with GLP1R^{SNAP/SNAP}, orthogonally labelled using BG-TMR, BG-JF646 or BG-Sulfo646 (all SNAP labels).

Together, these results show the specificity of the novel probes to label endogenous GLP1R/GIPR. The results are shown in FIG. 6-11.

### Example 2

HEK293T were transfected with Halo_GLP1R and/or SNAP-GIPR before labelling with Tirzepatide Cy3 (CH116) or Tirzepatide Cy5 (CH115). Counter-labelling Halo and SNAP was performed using 1 μM Halo-Sulfo549, Halo-Sulfo646 or 500 nM SBG-OG for 10 min at 37° C., cells were washed once, the image was acquired using in fluorobrite using a Nikon Ti-E base equipped with a 60× oil objective. Note clear co-localization of Tirzepatide Cy3 (CH116) and Tirzepatide Cy5 (CH115) with Halo_GLP1R and SNAP-GIPR, confirming probe specificity for both receptors. The results are shown in FIGS. 12-13.

In another experiment HEK293T (P154 (Halo-GLP1R):HEK293T) were stained with embodiments of the conjugates according to the present invention, namely Tirzepatide Cy3 (CH116) or Tirzepatide Cy5 (CH115) as antagonists of GLP1R/GIPR. HEK cells were seeded at 75000 cells/well on PLL-coated μ-well ibidi slide and transfected using JetPrime 400 ng. HEK cells were stained on the following day using 5 μM Hoechst3342, 500 nM CH115/CH116, 500 nM SBG-OG for 10 min at 37° C., cells were washed once, the image was acquired in fluorobrite at 60× oil.

### REFERENCES

1. Baggio, L. L. and D. J. Drucker, Biology of incretins: GLP-1 and GIP. Gastroenterology, 2007. 132(6): p. 2131-2157.
2. Samms, R. J., et al., GIPR Agonism Inhibits PYY-Induced Nausea-Like Behavior. Diabetes, 2022.
3. Mroz, P. A., et al., Optimized GIP analogs promote body weight lowering in mice through GIPR agonism not antagonism. Mol Metab, 2019. 20: p. 51-62.
4. Samms, R. J., et al., GIPR Function in the Central Nervous System: Implications and Novel Perspectives for GIP-Based Therapies in Treating Metabolic Disorders. Diabetes, 2021. 70(9): p. 1938-1944.
5. Bossart, M., et al., Effects on weight loss and glycemic control with SAR441255, a potent unimolecular peptide GLP-1/GIP/GCG receptor triagonist. Cell Metab, 2022. 34(1): p. 59-74.e10.
6. Killion, E., et al., Anti-obesity effects of GIPR agonists alone and in combination with GLP-1R agonists in preclinical models. Science Translational Medicine, 2018. 10(472).
7. Frías, J. P., et al., Tirzepatide versus Semaglutide Once Weekly in Patients with Type 2 Diabetes. N Engl J Med, 2021.385(6): p. 503-515.
8. Finan, B., et al., A rationally designed monomeric peptide triagonist corrects obesity and diabetes in rodents. Nat Med, 2015.21(1): p. 27-36.
9. Lean, M. E., et al., Tolerability of nausea and vomiting and associations with weight loss in a randomized trial of liraglutide in obese, non-diabetic adults. Int J Obes (Lond), 2014. 38(5): p. 689-97.
10. Tschöp, M. and R. DiMarchi, Single-Molecule Combinatorial Therapeutics for Treating Obesity and Diabetes. Diabetes, 2017. 66(7): p. 1766-1769.
11. Samms, R. J., M. P. Coghlan, and K. W. Sloop, How May GIP Enhance the Therapeutic Efficacy of GLP-1?Trends Endocrinol Metab, 2020. 31(6): p. 410-421.
12. Müller, T. D., et al., Anti-Obesity Therapy: from Rainbow Pills to Polyagonists. Pharmacol Rev, 2018. 70(4): p. 712-746.
13. Finan, B., et al., Unimolecular dual incretins maximize metabolic benefits in rodents, monkeys, and humans. Sci Transl Med, 2013.5(209): p. 209ra151.
14. Coskun, T., et al., LY3298176, a novel dual GIP and GLP-1 receptor agonist for the treatment of type 2 diabetes mellitus: From discovery to clinical proof of concept. Mol Metab, 2018. 18: p. 3-14.
15. Zhang, Q., et al., The glucose-dependent insulinotropic polypeptide (GIP) regulates body weight and food intake via CNS-GIPR signaling. Cell Metab, 2021.

1. A peptide conjugate comprising a peptide and a label,
wherein the peptide comprises an amino acid sequence of a protein ligand that binds at least one class B1 G protein-coupled receptor,
wherein the peptide is covalently linked to the label.

2. The peptide conjugate according to claim 1, wherein the protein ligand is an agonist, antagonist and/or co-agonist of at least one class B1 G protein-coupled receptor.

3. The peptide conjugate according to claim 1, wherein the protein ligand comprises an amino acid sequence with at least 70% identity, to an endogenous human protein ligand of at least one class B1 G protein-coupled receptor.

4. The peptide conjugate according to claim 1, wherein the protein ligand comprises a combination of sequences from multiple class B1 G protein-coupled receptor agonists.

5. The peptide conjugate according to claim 1, wherein the protein ligand is tirzepatide.

6. The peptide conjugate according to claim 1, wherein the protein ligand is LY3437943.

7. The peptide conjugate according to claim 1, wherein the protein ligand is GIP, or comprises a sequence of GIP or a fragment thereof.

8. (canceled)

9. The peptide conjugate according to claim 1, wherein the protein ligand is glucagon or a precursor protein thereof.

10. The peptide conjugate according to claim 1, wherein at least one class B1 G protein-coupled receptor is selected from the Glucagon-like subfamily, comprising Glucagon-like peptide 1 receptor (GLP-1R), glucose-dependent insulinotropic polypeptide receptor (GIPR), GLP-2R, GCGR and SCTR.

11. The peptide conjugate according to claim 1, wherein the label is selected from the group comprising a fluorescent label, a luminescent label, a nucleic acid label, a peptide label, an antibody or antigen-binding fragment thereof, a biotin label, a chromogenic label, an MRI label, a metal label or a radioactive label, and wherein the fluorescent label selected from the group consisting of cyanines, Janelia Fluors and rhodamines.

12. (canceled)

13. The peptide conjugate according to claim 1, wherein the peptide comprises a cysteine residue at its C-terminal and/or wherein the C-terminus of the peptide is amidated and/or wherein the peptide conjugate comprises a linker between the peptide and the label.

14.-17. (canceled)

18. The peptide conjugate according to claim 1, wherein the amino acid sequence of the protein ligand comprises one or more amino acid sequence variations, compared to an endogenous human sequence of the protein ligand, wherein the sequence variation comprises one or more chemical modifications, deletions, substitutions and/or insertions, wherein said substitutions and/or insertions are selected from proteinogenic amino acids, non-proteinogenic amino acids, naturally occurring amino acids or artificial amino acids.

19. The peptide conjugate according to claim 1, wherein the protein ligand is an agonist and/or co-agonist that is tirzepatide or GIP, and the tirzepatide or GIP amino acid sequence comprises at least one non-proteinogenic amino acid.

20. The peptide conjugate according to the preceding claim 19, wherein the at least one non-proteinogenic amino acid is or comprises 2-aminoisobutyric acid.

21. The peptide conjugate according to claim 19, wherein the tirzepatide does not comprise a conjugated fatty diacid module.

22. The peptide conjugate according to claim 1, wherein the peptide comprises or consist of an amino acid sequence according to SEQ ID NO 1-20, or an amino acid sequence of at least 80% identity thereto.

23. An in vitro method for assembling the peptide conjugate according to claim 1, comprising covalently linking the peptide to the label, and wherein the peptide comprises a cysteine residue at its C-terminal end.

24. An in vitro method for labelling and/or detecting the presence and/or localization of class B1 G protein-coupled receptors in a sample, comprising
contacting a sample with a peptide conjugate according to claim 1,
wherein the sample comprises at least one cell,
wherein the peptide conjugate binds to a class B1 G protein-coupled receptor present within the cell and/or the cell membrane of at least one cell, and
detecting the presence and/or localization of the peptide conjugate bound to a class B1 G protein-coupled receptor.

25. The method according to claim 24, which is for labelling class B1 G protein-coupled receptors in vitro.

26. A kit comprising:
the peptide conjugate according to claim 1, or
a peptide and a label, wherein the peptide comprises an amino acid sequence of a protein ligand of at least one class B1 G protein-coupled receptor, or comprises an amino acid sequence with least 70% identity to said protein ligand, wherein the peptide and the label are configured for covalent linkage.

27.-29. (canceled)


SEQUENCE LISTING (25 sequences)

SEQ ID NO: 1 - protein, 45 aa, synthetic construct
  SITE 2: note = Aib
  MOD_RES 45: note = S-Cy3-cysteine
  MOD_RES 45: note = cysteinamide
YXEGTFISDYSIAMDKIRQQDFVNWLLAQRGKKSDWKHNITQGGC

SEQ ID NO: 2 - protein, 45 aa, synthetic construct; identical to SEQ ID NO: 1
  SITE 2: note = Aib
  MOD_RES 45: note = S-Cy5-cysteine
  MOD_RES 45: note = cysteinamide

SEQ ID NO: 3 - protein, 39 aa, synthetic construct
  MOD_RES 39: note = S-Cy3-cysteine
  SITE 2: note = Aib
  SITE 13: note = Aib
  MOD_RES 39: note = cysteinamide
YXEGTFTSDYSIXLDKIAQKAFVQWLIAGGPSSGAPPPC

SEQ ID NO: 4 - protein, 39 aa, synthetic construct; identical to SEQ ID NO: 3
  MOD_RES 39: note = S-Cy5-cysteine
  SITE 2: note = Aib
  SITE 13: note = Aib
  MOD_RES 39: note = cysteinamide

SEQ ID NO: 5 - protein, 39 aa, synthetic construct
  SITE 2: note = Aib
  SITE 13: note = Aib
  MOD_RES 39: note = serinamide
YXEGTFTSDYSIXLDKIAQKAFVQWLIAGGPSSGAPPPS

SEQ ID NO: 6 - protein, 39 aa, synthetic construct; identical to SEQ ID NO: 3
  SITE 2: note = Aib
  SITE 13: note = Aib
  MOD_RES 39: note = cysteinamide

SEQ ID NO: 7 - protein, 39 aa, synthetic construct
  MOD_RES 13: note = alpha-methyl-L-leucine
  SITE 20: note = Aib
  SITE 2: note = Aib
  MOD_RES 39: note = serinamide
YXQGTFTSDYSILLDKKAQXAFIEYLLEGGPSSGAPPPS

SEQ ID NO: 8 - protein, 45 aa, synthetic construct; identical to SEQ ID NO: 1
  SITE 2: note = Aib
  MOD_RES 45: note = cysteinamide

SEQ ID NO: 9 - protein, 45 aa, synthetic construct; identical to SEQ ID NO: 1
  SITE 2: note = Aib

SEQ ID NO: 10 - protein, 42 aa, synthetic construct
  SITE 2: note = Aib
YXEGTFISDYSIAMDKIRQQDFVNWLLAQRGKKSDWKHNITQ

SEQ ID NO: 11 - protein, 42 aa, synthetic construct
  MOD_RES 42: note = glutamine amide
YAEGTFISDYSIAMDKIRQQDFVNWLLAQRGKKSDWKHNITQ

SEQ ID NO: 12 - protein, 42 aa, synthetic construct
YAEGTFISDYSIAMDKIHQQDFVNWLLAQKGKKNDWKHNITQ

SEQ ID NO: 13 - protein, 38 aa, synthetic construct
  SITE 2: note = Aib
  SITE 13: note = Aib
YXEGTFTSDYSIXLDKIAQKAFVQWLIAGGPSSGAPPP

SEQ ID NO: 14 - protein, 39 aa, synthetic construct; identical to SEQ ID NO: 7
  SITE 2: note = Aib
  SITE 20: note = Aib
  MOD_RES 13: note = alpha-methyl-L-leucine

SEQ ID NO: 15 - protein, 153 aa, Homo sapiens
MVATKTFALLLLSLFLAVGLGEKKEGHFSALPSLPVGSHAKVSSPQPRGPRYAEGTFISDYSIAMDKIHQQDFVNWLLAQKGKKNDWKHNITQREARALELASQANRKEEEAVEPQSSPAKNPSDEDLLRDLLIQELLACLLDQTNLCRLRSR

SEQ ID NO: 16 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 17 - protein, 29 aa, Homo sapiens
HSQGTFTSDYSKYLDSRRAQDFVQWLMNT

SEQ ID NO: 18 - protein, 37 aa, Homo sapiens
HDEFERHAEGTFTSDVSSYLEGQAAKEFIAWLVKGRG

SEQ ID NO: 19 - protein, 30 aa, Homo sapiens
HAEGTFTSDVSSYLEGQAAKEFIAWLVKGR

SEQ ID NO: 20 - protein, 31 aa, Homo sapiens
HAEGTFTSDVSSYLEGQAAKEFIAWLVKGRG

SEQ ID NO: 21 - DNA, 716 bp, Homo sapiens
  source 1..716: mol_type = other DNA
agcaggctcagaaggtccagaaatcaggggaaggagacccctatctgtccttcttctggaagagctggaaaggaagtctgctcaggaaataaccttggaagatggtggccacgaagacctttgctctgctgctgctgtccctgttcctggcagtgggactaggagagaagaaagagggtcacttcagcgctctcccctccctgcctgttggatctcatgctaaggtgagcagccctcaacctcgaggccccaggtacgcggaagggactttcatcagtgactacagtattgccatggacaagattcaccaacaagactttgtgaactggctgctggcccaaaaggggaagaagaatgactggaaacacaacatcacccagagggaggctcgggcgctggagctggccagtcaagctaataggaaggaggaggaggcagtggagccacagagctccccagccaagaaccccagcgatgaagatttgctgcgggacttgctgattcaagagctgttggcctgcttgctggatcagacaaacctctgcaggctcaggtctcggtgactctgaccacacccagctcaggactggattctgcccttcacttagcacctgcctcagccccactccagaatagccaagagaacccaaaccaataaagtttatgctaagtcgagcccattgtgaaaatttattaaaatgactactgagcactaa

SEQ ID NO: 22 - DNA, 1124 bp, Homo sapiens
  source 1..1124: mol_type = other DNA
acagagcttaggacacagagcacatcaaaagttcccaaagagggcttgctctctcttcacctgctctgttctacagcacactaccagaagacagcagaaatgaaaagcatttactttgtggctggattatttgtaatgctggtacaaggcagctggcaacgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacatgctgatggttctttctctgatgagatgaacaccattcttgataatcttgccgccagggactttataaactggttgattcagaccaaaatcactgacaggaaataactatatcactattcaagatcatcttcacaacatcacctgctagccacgtgggatgtttgaaatgttaagtcctgtaaatttaagaggtgtattctgaggccacattgctttgcatgccaataaataaattttcttttagtgttgtgtagccaaaaattacaaatggaataaagttttatcaaaatattgctaaaatatcagctttaaaatatgaaagtgctagattctgttattttcttcttattttggatgaagtaccccaacctgtttacatttagcgataaaattatttttctatgatataatttgtaaatgtaaattattccgatctgacatatctgcattataataataggagaatagaagaactggtagccacagtggtgaaattggaaagagaactttcttcctgaaacctttgtcttaaaaatactcagctttcaatgtatcaaagatacaattaaataaaattttcaagcttc

SEQ ID NO: 23 - DNA, 540 bp, Homo sapiens
  source 1..540: mol_type = other DNA
atgaaaagcatttactttgtggctggattatttgtaatgctggtacaaggcagctggcaacgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacatgctgatggttctttctctgatgagatgaacaccattcttgataatcttgccgccagggactttataaactggttgattcagaccaaaatcactgacaggaaa

SEQ ID NO: 24 - DNA, 87 bp, Homo sapiens
  source 1..87: mol_type = other DNA
cattcacagggcacattcaccagtgactacagcaagtatctggactccaggcgtgcccaagattttgtgcagtggttgatgaatacc

SEQ ID NO: 25 - DNA, 108 bp, Homo sapiens
  source 1..108: mol_type = other DNA
cacgatgaatttgagagacatgctgaagggacctttaccagtgatgtaagttcttatttggaaggccaagctgccaaggaattcattgcttggctggtgaaaggccga